(19) United States
(12) Patent Application Publication
(10) Pub. No.: US 2014/0322193 A1
(43) Pub. Date: Oct. 30, 2014

(54) Therapies for Disorders of the Cornea and Conjunctiva

(71) Applicant: Michael Kaleko, Potomac, MD (US)

(72) Inventor: Michael Kaleko, Potomac, MD (US)

(21) Appl. No.: 14/362,330

(22) PCT Filed: Dec. 1, 2012
(86) PCT No.: PCT/US12/67493

§ 371 Date: Jun. 2, 2014

Related U.S. Application Data
(60) Provisional application No. 61/566,011, filed on Dec. 2, 2011.

(51) Int. Cl. A61K 38/46; A61K 31/7076; A61K 31/715; A61K 31/47; A61K 31/4439

(52) U.S. Cl. CPC A61K 38/46; A61K 31/47; A61K 31/4439; A61K 31/715; A61K 31/7076
USPC 424/94.6; 514/313; 514/340; 514/54; 514/47

(57) ABSTRACT

Unexpectedly, disorders of the cornea and conjunctiva are found to be caused or to be exacerbated, at least in part, by extracellular ATP and P2X7 receptor activation. Therapeutic compositions for topical administration to the eye for the treatment of disorders of the cornea and conjunctiva include an entity that inhibits P2X7 receptor function, reduces the effective concentration of extracellular ATP, or both.

### FIELD OF THE INVENTION

The present invention relates to ophthalmic compositions for topical delivery to the eye that attenuate P2X7 receptor activation and/or diminish the concentration of extracellular ATP for treating disorders of the cornea and conjunctiva (DOCC).

### BACKGROUND OF THE INVENTION

The specialized tissues at the front of the eye protect the eye from the environment and include the cornea, conjunctiva, eyelids and associated secretory glands, and lacrimal gland. The cornea is the clear tissue that refracts and focuses light. The conjunctiva is the membrane that protects and seals off the front of the eye, preventing foreign bodies from moving behind the eye. The eyelids close to keep the front of the eye moist and, by moving tears, wash away debris that lands on the eye. The lacrimal gland and associated ducts deliver the aqueous portion of the tears to the ocular surface and ensure proper tear drainage from the surface. Importantly, the tissues of the front of the eye collaborate to produce the appropriate amount of tears, ensure flow across the surface, and ensure drainage. The tears wash the front surfaces, remove debris, keep the tissues moist, lubricate the movement of the eyelids over the ocular surface, and provide proteins and immune mediators to protect the eye from invading organisms including viruses, bacteria, fungi, and parasites.

The cornea is composed of an outer layer of epithelial cells, a fibrous layer, and an inner monolayer of endothelial cells. The corneal shape and clarity are critical to vision. Accordingly, the cornea must be maintained free of blood vessels. The conjunctiva is composed of connective tissue that contains blood vessels and lymphatics. It, too, has an outer layer of epithelial cells. The conjunctival membrane is juxtaposed to the sclera, the white part of the eye, in the front of the eye (bulbar conjunctiva), but then reflects back and lines the inside of the eyelids (tarsal conjunctiva) to the eyelid margin. The conjunctiva thus forms a protective shield separating the front and back of the eye.

These tissues respond to changing environmental stresses in a well-regulated and coordinated fashion. For example, tear formation and flow are controlled by neural loops with afferent neurons in, for example, the cornea, and efferent neurons in, for example, the lacrimal glands. Neural loops also control the blink reflex to modulate the flow of tears across the surface. The tear layer is very complex and is actually composed of three layers. The innermost layer, against the cornea and conjunctiva, is produced by conjunctival goblet cells and contains mucinous proteins that ensure even wetting of the corneal surface. The middle layer, produced by the lacrimal glands and conjunctiva, is aqueous and provides nourishment to the ocular surface. The outer layer, secreted by the tarsal glands, including the meibomian glands, is composed of lipids and serves to prevent rapid evaporation.

This delicate and complex system must function properly to protect the eye and maintain vision. Not surprisingly then, there are many pathological conditions that disrupt its function and which cause signs and symptoms referable to the cornea and conjunctiva. Several common pathological conditions include allergic diseases, injuries, blepharitis, and dry eye.

Allergic eye conditions are extremely common and include seasonal allergic conjunctivitis, perennial allergic conjunctivitis, atopic keratoconjunctivitis, vernal keratoconjunctivitis, giant papillary conjunctivitis, and contact allergies. An alternate nomenclature refers to the seasonal and perennial conditions as intermittent and persistent allergic conjunctivitis, respectively. Keratoconjunctivitis and blepharoconjunctivitis refer to conditions affecting the cornea and eyelids, respectively, in addition to the conjunctiva.

Seasonal and perennial conjunctivitis represent approximately 25-50% of cases, are caused by airborne allergens, and, for the most part, are mediated by mast cell degranulation with the release of histamine, tryptase, eotaxin, and eosinophilic cationic protein. Symptoms include itching, redness, chemosis, and tearing, which lead to conjunctival redness and edema.

Atopic keratoconjunctivitis is a chronic ocular allergy primarily in the adult population. Symptoms include itching, burning, tearing, and erythematous and swollen eyelids. It can progress to corneal scarring, opacity, and neovascularization. The eyelids and conjunctiva can exhibit scarring. This condition has a complex etiology involving both the innate and adaptive arms of the immune system. Vernal keratoconjunctivitis represents 0.5% of allergic conditions and usually occurs in children. Symptoms include severe ocular itching, tearing, photophobia, and mucus discharge. The presentation is similar to that of atopic keratoconjunctivitis; however, vernal keratoconjunctivitis exhibits a unique feature of giant cell papillae on the tarsal conjunctiva. Vernal keratoconjunctivitis can lead to a non-healing corneal ulcer and corneal scarring. This condition also has a complex immunological etiology.

Giant papillary conjunctivitis is caused by the presence of a foreign body such as a contact lens, prosthesis, or corneal sutures. Allergic responses are directed towards adsorbed foreign particles or proteins or to contact lens solutions.

Contact allergy is primarily due to allergen exposure from cosmetics or chemicals. It is predominantly a Th1-mediated delayed hypersensitivity response with symptoms of itching and conjunctival hyperemia. The surrounding skin can also demonstrate contact dermatitis.

Treatments for allergic eye diseases include eye drops that deliver antihistamines, mast cell stabilizers (e.g. sodium cromoglycate), and nonsteroidal anti-inflammatory agents. The more severe allergic conditions, which can lead to blindness, can require treatment with corticosteroids and immunomodulatory agents including cyclosporine, tacrolimus, mycophenolate mofetil, leflunomide, rapamycin, laquinimod, and infliximab. The allergic eye diseases continue to cause significant ocular morbidity and would benefit from the introduction of new therapeutic modalities.

Allergic eye diseases are summarized in Journal of Asthma and Allergy 2010 3:149 and Recent Pat Inflamm Allergy Drug Discov 2011 5:26.

Injuries to the cornea are quite common and can be caused, for example, by thermal burns, chemical burns, physical abrasion from foreign bodies, overuse of contact lenses, reaction to contact lens solutions, excessive exposure to sunlight or sunlamps, ocular surgeries, corneal transplants, and infections. Chemical burns from strong alkaline solutions can be particularly problematic. Symptoms of corneal injury include pain, photophobia, blurred and decreased vision, redness, swollen eyelids, and excessive tearing. Treatment involves removing the foreign object, but is otherwise mostly supportive. Pain medication and antibiotics may be warranted. Many wounds heal well; however, severe wounds may require surgery or corneal transplant. One serious complication is corneal neovascularization, which can impair vision.

Blepharitis is an inflammatory condition of the eyelids, with anterior blepharitis affecting the lid margins, where the eyelashes are attached, and posterior blepharitis affecting the inner edge of the eyelid that comes in contact with the eye. Common causes include skin conditions and bacterial infections. Signs and symptoms include a gritty or burning sensation, excessive tearing, itching, red and swollen eyelids, dry eyes, or crusting of the eyelids. More severe cases can lead to blurred vision, missing or misdirected eyelashes, dry eye, and corneal inflammation. Treatment usually consists of attention to hygiene and, where appropriate, antibiotics. While blepharitis usually does not threaten eyesight, the condition can be persistent.

Dry eye is also known by the terms dry eye syndromes, ocular surface disease, deficient tear syndrome, and keratoconjunctivitis sicca. It is estimated that 14% of adults over the age of 40 and up to 19% of people over 80 have dry eye. This translates to more than 20 million Americans. There are many causes, most of which can be separated into two main categories, aqueous deficient dry eye and evaporative dry eye (Care of the Patient with Ocular Surface Disorders 2011 American Optometric Association). Aqueous deficient dry eye could be caused, for example, by insufficient lacrimal gland secretion, whereas evaporative dry eye could be caused, for example, by meibomian gland disorders with an insufficient lipid coating to prevent tear evaporation. Within each of these categories there are multiple etiologies. Symptoms of dry eye include pain, a stinging, burning, or scratchy sensation, increased eye irritation from smoke or wind, stringy mucus in or around the eyes, eye fatigue, sensitivity to light, blurred vision, difficulty wearing contact lenses, and redness. Potential therapies include eye drops to wet the eyes (artificial tears) and prescription inserts to provide artificial tears. Some, but not all cases, respond to prescription antibiotics to decrease inflammation and prescription anti-inflammatory and immunosuppressive drugs including cyclosporine and corticosteroids (Arq Bras Oftalmol 2008 71:89). These latter two therapies can be associated with significant side effects. In rare cases, surgery or plugs to block the tear drainage can be considered. In general, dry eye is a prevalent condition with inadequate therapies and an ongoing unmet medical need.

Disorders of the cornea and conjunctiva (DOCC), including but not limited to those described above, affect a large number of people and range in severity from mild irritation to sight-threatening. Although they share an overlapping set of signs and symptoms, these disorders have very diverse etiologies. DOCC remain a significant cause of ocular morbidity and would substantially benefit from new therapeutic entities.

The current invention describes a novel therapeutic modality with broad applicability for the treatment of DOCC; that is, inhibiting the P2X7 receptor or diminishing the concentration of its major activating ligand, extracellular ATP.

Adenosine 5′-triphosphate (ATP) is widely known as the major energy storage molecule in the cell. Accordingly, intracellular ATP levels are maintained in the millimolar range. In the past two decades, there has been an increasing appreciation that extracellular ATP is involved in cell signaling (Molecular Pharmacology 2003 64:785). In contrast to the high intracellular levels, concentrations of ATP outside the cell are usually quite low, in the nanomolar to micromolar range. In certain circumstances ATP is released from cells. Such circumstances range from physiologic intercellular signaling and synaptic transmission to inflammation, increased extracellular pressure, osmotic stress, and mechanical stress. Mechanisms of ATP secretion include release of exocytotic vesicles and secretion via ion channels, pannexin and connexin hemichannels, and membrane pores. ATP is also released in large quantities by dying cells. ATP can be synthesized outside the cell by enzymes including adenylate kinase. Extracellular ATP levels are, in part, controlled via degradation by a series of enzymes including the ecto-NTPDases, 5′ ecto-nucleotidase, adenosine deaminase (ADA), and purine nucleoside phosphorylase (PNP), which convert ATP to adenosine 5′-diphosphate (ADP), adenosine 5′-monophosphate (AMP), adenosine, inosine, and hypoxanthine.

Extracellular ATP and its degradation products activate cell membrane, purinergic receptors called P1 and P2 receptors. The four P1 receptors, A1, A2a, A2b, and A3, are triggered by adenosine. P2 receptors are triggered by ATP and, in some cases, other nucleotides. There are two types of P2 receptors, P2Y and P2X. The eight P2Y receptors are metabotropic GPCRs, and the seven P2X receptors function as nonselective ion channels. Splice variants and heteromeric receptors further increase the complexity of this signaling system. Subsets of the P1 and P2 receptors are found on most cells, where they mediate intercellular and autocrine communication in a receptor and cell type specific manner. Purinergic signaling is reviewed in Biochimica et Biophysica Acta 2008 1783:673, Science Signaling 2010 3:1, and Advances in Pharmacology 2011 61:333.

Among the P2X receptors, P2X7 is unique. All P2X receptors with the exception of P2X7 are activated by micromolar levels of extracellular ATP. In contrast, the P2X7 receptor requires approximately one to two orders of magnitude higher levels of ATP for activation. Importantly, in some cell types, the P2X7 receptor can change function based on the level and duration of ATP exposure. Whereas the initial exposure to ATP activates a nonselective cation channel, increasing durations of exposure can lead to the formation of a large pore, through which molecules as large as 900 daltons can pass. Activation of the P2X7 receptor can lead to cell death through caspase activation and lysis.

P2X7 receptors are found on many cell types including epithelial cells, endothelial cells, neuronal and glial cells, and cells of hematopoietic origin including monocytes, macrophages, dendritic cells, and some B and T lymphocytes. In immune cells, stimulating the P2X7 receptor generally enhances a pro-inflammatory response leading to the release of IL-1β and IL-18. Pore formation can release additional ATP and can ultimately lead to cell death. In the nervous system, stimulating the P2X7 receptor can trigger astrocyte activation, alter neuronal function, and enhance nociception and hyperalgesia (FASEB J. 2010 24:337).

### SUMMARY OF THE INVENTION

The present invention envisions an unexpected role for ATP and purinergic signaling in protecting the cornea and conjunctiva. That is, ATP is envisioned to participate with the known neural pathways in regulating tear production, tear composition, and the blink reflex. ATP is also envisioned to participate in the maintenance of corneal integrity and ocular immune defenses.

The present invention envisions that ATP is secreted onto the surface of the cornea and conjunctiva as a result of mechanical stress on the corneal and conjunctival epithelial cells. Such stress can come from movements of the eye and the eyelids. Specifically, as the eye moves and the eyelids open and close, the tarsal conjunctival epithelial cells slide past the bulbar conjunctival and corneal epithelial cells. This process is normally well lubricated by tears, which minimizes the mechanical stress on these cells and thereby minimizes the release of extracellular ATP.

Additionally, the present invention envisions that ATP normally signals within a negative feedback loop. If the lubrication should become inadequate, or if irritating debris should land on the eye, the epithelial layers moving past one another would be subjected to increased mechanical stress, which would increase ATP secretion. The ATP could then serve to increase nociception and to stimulate the neural reflexes to increase tear production and blinking. The ATP could also stimulate epithelial migration (IOVS 2008 49:4384) and, if needed, an immune response to maintain corneal integrity. With increased tear production and blinking, the lubrication would return to normal and irritating debris would be washed away. The mechanical stress would decrease. ATP secretion would abate, and ecto-nucleotidases would degrade any excess ATP.

The present invention envisions that if these physiologic responses are unable to re-establish the normal, well lubricated ocular surfaces, then ATP signaling becomes detrimental. Such situations include, but are not limited to, insufficient tear production, allergic reaction, injury, and infection. In these contexts, the mechanical stress does not resolve and ATP secretion continues unabated. Rising ATP levels ultimately trigger P2X7 receptors on the corneal and conjunctival epithelial cells as well as the underlying fibroblasts. P2X7 receptor activation can lead to pore formation, caspase activation, corneal and conjunctival cell death, and epithelial cell sloughing from the ocular surface. The resultant damage to the surface of the eye would be associated with pain. Thus, the present invention envisions that dysregulation of ATP signaling is a common etiologic factor in many disorders of the cornea and conjunctiva.

The present invention envisions that the pathology caused by P2X7 receptor activation could become self-sustaining via several potential mechanisms. First, pore formation and cell lysis would lead to the continued release of ATP. Second, activation of the P2X7 receptor could stimulate cytokine release, which would increase pain and recruit inflammatory cells. Moreover, activation of P2X7 receptors on the infiltrating inflammatory cells could serve to ramp up an inflammatory reaction. Third, damage to the meibomian glands and conjunctival goblet cells could exacerbate the pathology by diminishing tear production and quality. In extreme cases, P2×7-mediated processes could result in corneal ulceration, neovascularization, and opacity.

The present invention also envisions that, in contexts similar to those that stimulate ATP release, nicotinamide adenine dinucleotide (NAD) is also released from cells. Extracellular NAD serves as a substrate for ADP-ribosylation of the P2X7 receptor, which leads to receptor activation. Thus, like extracellular ATP, extracellular NAD can play a role in exacerbating DOCC.

While not intending to be limited by the specific mechanisms envisioned herein, according to the present invention, P2X7 receptor signaling is a common denominator that initiates and propagates damage to the cornea and conjunctiva in many disorders of the cornea and conjunctiva, despite the diverse etiologies of these disorders. The invention describes administration of compositions that attenuate P2X7 receptor activation or diminish the concentration of its activating ligands as a means of mitigating damage to the cornea and conjunctiva and treating the signs and symptoms of DOCC.

The compositions of the present invention are designed to be delivered topically to the front of the eye, via, for example, eye drop formulations. The dosages and frequency of administration will be readily determined by one skilled in the art to diminish the signs and symptoms of pathology without incurring undue adverse effects. The compositions of the present invention can be used as single agents or in combination with other ocular medications. The compositions of the present invention can be combined with known drugs for treating ocular diseases into a single composition.

In one embodiment, the compositions of the current invention comprise an inhibitor of the P2X7 receptor. Such an inhibitor may be a non-specific inhibitor that also inhibits the actions of other receptors and proteins or it can be a specific inhibitor that has much greater potency for inhibiting the P2X7 receptor than other receptors and proteins. The inhibitor of the P2X7 receptor can be one that transiently inhibits the P2X7 receptor; that is, only when the inhibitor is present at therapeutically effective levels. Alternatively, the P2X7 inhibitor can be an irreversible inhibitor; that is, one that permanently inactivates the P2X7 receptor such that inhibition persists after the inhibitor is no longer present.

In one embodiment, the compositions of the present invention comprise molecules that degrade extracellular ATP such that the levels of extracellular ATP and the pathology caused by extracellular ATP decrease. The dosage and frequency of administration for compositions that degrade extracellular ATP will be readily determined clinically and empirically by one skilled in the art with the dosage and frequency of administration titrated to diminish the signs and symptoms of ocular pathology without incurring undue adverse effects. Without intending to be limited by a specific mechanism, the present invention envisions diminishing extracellular ATP to levels that have minimal or no effect on the P2X7 receptor. The compositions of the present invention can comprise enzymes of the NTPDase family, which degrade ATP to ADP and AMP. The compositions can comprise 5′-nucleotidase, which degrades AMP to adenosine. The compositions can comprise adenosine deaminase, which degrades adenosine to inosine. The compositions can comprise purine nucleoside phosphorylase, which degrades inosine to hypoxanthine. The compositions of the present invention can comprise enzymes that are soluble proteins. Whereas, four members of the NTPDase family and 5′ ecto-nucleotidase are known to be membrane proteins, methods are described in the art to produce recombinant forms that are soluble proteins and that maintain sufficient biological activity and stability for therapeutic efficacy. Soluble forms of human NTPDase 1 (CD39) and human 5′ ecto-nucleotidase (CD73) have been described in the literature (J. of Clin. Invest. 1998 101:1851, U.S. Pat. No. 7,264,809, and Molecular Pain 2010 6:20).

In one embodiment, the proteins of the current compositions are not human proteins and can include, for example, enzymes such as apyrase derived from potatoes or 5′ nucleotidase derived from snake venom. In some embodiments, a protein of the invention is a human protein or a variant thereof.

Whereas the invention focuses on diminishing the effects of extracellular ATP on P2X7 signaling, a second molecule, NAD, is also released from cells in parallel with ATP. NAD serves as a substrate for enzymes of the ecto-ADP-ribosyltransferase (ART) family, which transfer ADP-ribosyl groups to the P2X7 receptor, and, in doing so, activate the receptor. This activation pathway is down-regulated by enzymes of the NAD-glycohydrolase family, particularly CD38, which hydrolyze NAD. Therefore, compositions of the present invention also comprise inhibitors of NAD-mediated activation of the P2X7 receptor.

Formulations for the manufacture of medications for topical delivery to the eye are known in the art. Formulations for the present invention comprise a pharmaceutically acceptable carrier that is compatible with the surface of the eye. A formulation may also promote the efficacy and stability of one or more active substances such as a P2X7 inhibitor or ATP degrading enzyme. In one embodiment, compositions for topical delivery to the eye comprise eye drops. In another embodiment, compositions for topical delivery to the eye comprise a colloidal suspension, cream, or ointment. In one embodiment, the eye drop formulations comprise simple salt solutions and buffers. In one embodiment, the eye drop formulations comprise agents to increase viscosity, such as carboxymethylcellulose or a gelling agent to prolong the time that the medications remain on the surface of the eye. In one embodiment, formulations for topical administration comprise emulsifiers to promote the solubility of the P2X7 inhibitor. In one embodiment, formulations for topical administration comprise cyclodextrins. In one embodiment, formulations for topical delivery comprise a preservative. In an embodiment, formulations for topical delivery do not contain a preservative. In some embodiments, the active substances of the present invention are delivered to the eye using a mechanical device such as a contact lens.

The present invention provides compositions, formulations, and methods for treating disorders of the cornea and conjunctiva (DOCC). Such DOCC comprise any disorder with pathology that manifests, at least in part, with signs and/or symptoms referable to the cornea and/or conjunctiva. Such disorders include those that alter the function and/or the histology of the cornea and/or conjunctiva. Such disorders include those associated with signs and/or symptoms including pain, burning, stinging, foreign body sensation, itching, excessive lacrimation, chemosis, redness, dryness, edema, photophobia, blurred vision, mucus discharge, purulent discharge, neovascularization, corneal opacity, corneal ulceration, corneal scarring, or blindness. DOCC include but are not limited to allergic eye diseases, blepharitis, dry eye syndromes, and ocular injuries including those caused by burns, foreign bodies, contact lenses, contact lens solutions, surgery, or infection.

The present invention further provides an article of manufacture comprising packaging material and a pharmaceutical agent together with an acceptable pharmaceutical carrier contained within said packaging material, wherein said packaging material comprises a label that indicates said pharmaceutical may be administered for a sufficient term and at a sufficient dose for treating DOCC, and wherein said pharmaceutical agent is an inhibitor of the P2X7 receptor, a molecule that diminishes the extracellular concentration of ATP, or both.

### BRIEF DESCRIPTION OF THE FIGURE

The following description of the FIGURE and the respective drawing are non-limiting examples that depict various embodiments that exemplify the present invention.

FIG. 1 demonstrates efficacy in a murine model of dry eye. Botulinum B toxin was injected into mouse lacrimal glands to induce dry eye. In this model, the mice develop corneal ulcerations during the ensuing two weeks, which are quantified by fluorescein staining. Four cohorts of mice were treated twice daily with eye drops, one with formulation only and three with P2X7 inhibitors. A negative control cohort received no eye drop treatment. The results showed that one P2X7 inhibitor, oxATP, was remarkably effective in preventing pathology. No fluorescein staining was seen in any eye treated with oxATP eye drops.

### DETAILED DESCRIPTION OF THE INVENTION

The present invention relates to compositions comprising an inhibitor of the P2X7 receptor or any molecule that attenuates the normal or excessive functioning of the P2X7 receptor, an enzyme that degrades ATP or any molecule that reduces the effective extracellular concentration of ATP or reduces the capacity of ATP to activate the P2X7 receptor, or both. The present invention describes the topical use of such compositions to mitigate damage to the cornea and conjunctiva and to treat DOCC. The present invention further relates to compositions comprising an inhibitor of NAD-mediated P2X7 receptor activation and the topical use of such compositions to mitigate damage to the cornea and conjunctiva and to treat DOCC.

The invention relates to compositions that prevent functioning of the P2X7 receptor. Hence, inhibitors, antagonists and the like are used herein to describe a compound or composition that obtains a diminution or reduction of P2X7 function. Also included are antibodies, or antigen-binding portions thereof, which may, for example, specifically bind to the extracellular domain of the P2X7 receptor or which may, for example, only sterically prevent the P2X7 ligand from properly engaging the P2X7 receptor at the cell surface. Hence, while inhibitors are exemplified in the disclosure, any entity that reduces P2X7 receptor activity can be used in the practice of the present invention.

The invention relates to compositions that prevent the signaling function of extracellular ATP. Hence, compounds, enzymes and the like that degrade, alter or reduce the level of ATP can be used in the practice of the method of interest. However, any molecular entity that prevents extracellular ATP from engaging a cytotoxic purinergic receptor, such as an antibody that specifically binds ATP or any other molecule that entraps or sequesters ATP, is contemplated to be included as compositions that can be used in the practice of the method of interest, so long as effective levels of ATP are reduced. Hence, while enzymes that degrade ATP are exemplified herein, any entity that reduces ATP signaling can be used in the practice of the instant invention.

The invention relates to compositions that diminish NAD-mediated activation of the P2X7 receptor. Hence compounds that degrade NAD or diminish its capacity to serve as a substrate for ecto-ADP-ribosyltransferase reactions, or molecules that inhibit the function of ecto-ADP-ribosyltransferases are contemplated to be included as compositions that can be used in the practice of the method of interest.

As used herein, unless the context clearly indicates, words used in the singular include the plural, and words used in the plural include the singular. As used herein, unless the context clearly indicates, the article, “the,” is not limiting. As used herein, unless the context clearly indicates, the term, “include,” has the meaning, “include, but is/are not limited to,” or, “comprise.”

As used herein, the phrase, “disorders of the cornea and conjunctiva,” and the acronym, “DOCC,” comprise any disorder with pathology that manifests, at least in part, with signs and/or symptoms referable to the cornea and/or conjunctiva. Such disorders include those that alter the function and/or the histology of the cornea and/or conjunctiva. Such disorders include those characterized by epithelial cell death, inflammation, and inflammatory infiltrates. Such disorders include those associated with signs and/or symptoms including pain, burning, stinging, foreign body sensation, itching, excessive lacrimation, chemosis, redness, dryness, edema, photophobia, blurred vision, mucus discharge, purulent discharge, corneal neovascularization, corneal opacity, corneal ulceration, corneal scarring, or blindness. Such disorders include those associated with diagnostic abnormalities including a reduced tear breakup time, decreased wetting on Schirmer testing, and increased dye staining. Staining of the cornea with a dye (e.g. fluorescein) is a common means of clinically assessing corneal damage.

As used herein, many disorders of the cornea and conjunctive can be categorized as: 1) allergic disorders of the cornea and conjunctiva; 2) corneal and conjunctival disorders related to injury; 3) disorders associated with blepharitis; 4) disorders related to dry eye; and 5) corneal and conjunctival disorders related to causes other than allergy, injury, blepharitis, and dry eye.

As used herein, “allergic disorders of the cornea and conjunctiva,” refer to: seasonal (also called intermittent) allergic conjunctivitis and keratoconjunctivitis; perennial (also called persistent) conjunctivitis and keratoconjunctivitis; vernal keratoconjunctivitis; atopic keratoconjunctivitis; giant papillary conjunctivitis; contact allergy; acute and chronic ocular allergies; and non-allergic eosinophilic conjunctivitis.

As used herein, “corneal and conjunctival disorders related to injury,” refer to disorders of the cornea and conjunctiva related to: trauma; lacerations; foreign bodies; burns; thermal injuries; UV burns secondary to sunlight, sunlamps, or arc welders; gamma irradiation; chemical injuries; post-surgical complications due to refractive, cataract, prosthesis, or other surgeries; corneal abrasions; corneal ulcers; contact lenses; contact lens solutions; the use of eye drops; the use of topical glaucoma medications; and bacterial, viral, fungal, or parasitic infections.

As used herein, “disorders associated with blepharitis,” refer to: blepharospasm, ulcerative blepharitis; squamous blepharitis; hordeolum; hordeolum externum; hordeolum internum; infection of the meibomian glands; abscess or furuncle of the eyelid; chalazion; meibomian cyst; noninfectious dermatoses of the eyelid; eczematous dermatitis of the eyelid; parasitic infestation of the eyelid; and inflammation of the eyelid.

As used herein, “disorders related to dry eye,” refer to: mucin deficiency; goblet cell deficiency; disorders of the lacrimal system; congenital alacrima; congenital dysautonomia; disorders of the lacrimal gland; dacryops; tear film insufficiency; dry eye syndrome; age-related dry eye; keratoconjunctivitis sicca; Sjogren's disease; deficient aqueous tear production due Sjogren's or non-Sjogren's lacrimal gland dysfunction; dacryoadenitis; deficient tear production due to medication side effects; deficient tear production due to hormone deficiency, hormone replacement, birth control pills, menopause, pregnancy, elevated levels of estrogen or prolactin, or testosterone deficiency; facial nerve paralysis; increased evaporative loss due to blepharitis with meibomian gland dysfunction, exposure, and other factors including blink abnormality, and lid abnormality; dysfunctional tear film with anterior or posterior lid margin disease; dysfunctional tear distribution due to conjunctivochalasis, lid and lash malpositions, elevated surface lesions, reduced or incomplete blinking; dysfunctional tear film without lid margin disease; and deficient tear distribution due to ocular surface abnormalities.

Examples of medications that can cause DOCC include: antihypertensives (e.g. diuretics, adrenergic antagonists, and β-blockers); antihistamines (especially first generation H-1 inhibitors); medications that have anti-cholinergic effects (e.g. tricyclic antidepressants, phenothiazines, etc.); and hormone replacement therapy (e.g. estrogen and progesterone). Examples of disorders that cause mucin deficiency include: ocular cicatricial pemphigoid; Stevens Johnson Syndrome; trachoma; vitamin A deficiency; and facial nerve paralysis. Examples of disorders associated with a blink abnormality or lid abnormality include: Bell's palsy; lagophthalmos; thyroid-related eye disease; lid trauma; ptosis; trichiasis; and madarosis. Examples of anterior and posterior lid margin disease include: anterior blepharitis due to eczema, infection, and seborrhea; staphylococcal blepharitis; seborrheic blepharitis (also called squamous blepharitis) associated with scalp, face, and eyebrow seborrheic dermatitis as well as rosacea, acne vulgaris, and Malassezia yeast infection; meibomian seborrheic blepharitis; seborrheic blepharitis with secondary meibomianitis; meibomian keratoconjunctivitis; angular blepharitis; demodicosis; and posterior blepharitis, which can be associated with meibomian gland dysfunction and abnormal tear film quality.

As used herein, “corneal and conjunctival disorders related to causes other than allergy, injury, blepharitis, and dry eye,” refer to: scleritis; iritis; uveitis; corneal epitheliopathies; genetic diseases and idiopathic diseases of the cornea and/or conjunctiva; keratoconus; corneal graft failure; corneal graft versus host disease; and ocular manifestations of systemic disorders with signs and symptoms referable to the cornea and/or conjunctiva.

As used herein, “disorders of the cornea and conjunctiva,” and, “DOCC,” include uveitis and iritis. While uveitis and iritis exhibit corneal and conjunctival manifestations, the pathology associated with these disorders is also directed to structures within the eye. As used herein, “uveitis,” and, “iritis,” refer to the corneal and conjunctival pathologies as well as to the intraocular pathologies associated with these disorders. As used herein, “uveitis,” refers to anterior, intermediate, or posterior uveitis and to autoimmune uveitis or uveitis of a non-autoimmune etiology such as infection.

A review of disorders with signs and symptoms referable to the cornea and conjunctiva can be found in Care of the Patient with Ocular Surface Disorders 2011 American Optometric Association, which is incorporated herein by reference in entirety.

Whereas the above examples of DOCC are meant to impart an understanding of the scope of the present invention, the examples and listing are not intended to provide an exhaustive list of all diseases or abnormalities referable to the cornea and conjunctiva. It is recognized that those skilled in the art can diagnose a specific disease or abnormality that is not included in the above description, but that would nonetheless constitute a disorder of the cornea and conjunctiva according to the present invention.

The present invention relates to the novel and unexpected discovery that the pathology associated with DOCC is caused and/or is exacerbated, at least in part, by extracellular ATP and P2X7 receptor activation. More specifically, the signs and symptoms of DOCC are worsened by extracellular ATP and P2X7 receptor activation, which act locally on the cornea and conjunctiva to damage these tissues. Accordingly, topical administration of compositions that reduce the concentration of extracellular ATP or inhibit the function of the P2X7 receptor can be used to mitigate damage to the cornea and conjunctiva and to treat DOCC.

As used herein, the phrase, “mitigate damage to the cornea and conjunctiva,” means to diminish any level of, slow the progression of, or prevent the onset of any aspect of corneal and/or conjunctival pathology and/or dysfunction. Such pathology and dysfunction can be readily assessed by clinicians skilled in the art through physical examination and laboratory analyses, which can include, for example: biomicroscope exam; tear analyses for quantity, quality, osmolarity, breakup time, IgE levels, cytokine levels, and lactoferrin levels; corneal and conjunctival staining with fluorescein and lissamine green; conjunctival impression cytology; meibomian gland expression; and conjunctival biopsy.

As used herein, the verb, “treat,” means to diminish any level of, slow the progression of, or prevent the onset of any sign or symptom of DOCC including pain, burning, stinging, foreign body sensation, itching, excessive lacrimation, chemosis, redness, dryness, edema, photophobia, blurred vision, mucus discharge, purulent discharge, corneal neovascularization, corneal opacity, corneal ulceration, corneal scarring, and blindness. Additional signs and symptoms of DOCC can include injected conjunctiva, floaters, headaches, loss of peripheral vision, flashes of light, dilated ciliary vessels, presence of cells and flare in the anterior chamber, and keratic precipitates on the posterior surface of the cornea.

As used herein, “P2×7,” and, “P2X7 receptor,” are used interchangeably and refer to all mammalian P2X7 receptors including the human P2X7 receptor and including all allelic and splice variants of the P2X7 receptor. The phrase, “P2X7 monomer,” refers to the individual monomers that compose the homomeric P2X7 receptor. As used herein, the phrase, “P2X7 receptor,” further includes any heteromeric receptor in which at least one of the monomers is a P2X7 monomer.

As used herein, the terms, “agonist,” and, “antagonist,” in reference to a receptor refer to molecules that will activate and inhibit the function of the receptor, respectively, as measured by a downstream effect of the receptor.

As used herein, the terms, “antagonist,” and, “inhibitor,” are synonyms and are used interchangeably. Accordingly, the phrases, “inhibitor of the P2X7 receptor,” “P2X7 receptor inhibitor,” “antagonist of the P2X7 receptor,” and, “P2X7 receptor antagonist,” all refer to an entity that inhibits the P2X7 receptor or inhibits P2X7 receptor function.

As used herein, the phrase, “irreversible inhibitor,” refers to an inhibitor that acts upon a P2X7 receptor to permanently inhibit said receptor. Irreversible inhibitors of the present invention function by covalently modifying and permanently altering the structure of a P2X7 receptor. As used herein, the phrase, “reversible inhibitor,” refers to an inhibitor that can dissociate from a P2X7 receptor thereby enabling said receptor to regain function. For the purposes of the present invention, any inhibitor that is not irreversible is considered reversible.

As used herein, the verb, “activate,” in reference to a receptor means to increase the receptor's activity by at least 5%, at least 10%, at least 15%, or more above a baseline or reference level of activity as measured by a downstream effect of the receptor.

As used herein, the verb, “inhibit,” in reference to a receptor means to diminish the activity of the receptor by at least 5%, at least 10%, at least 15%, or more below a baseline or reference level of activity, such as, at a given concentration of agonist, as measured by a downstream effect of the receptor.

Commonly measured downstream effects for the P2X7 receptor include Ca⁺⁺ influx and pore formation. Commonly used agonists for the P2X7 receptor include ATP and the more potent agonist, 2′(3′)—O-(4-benzoylbenzoyl)adenosine 5′-triphosphate (BzATP). Assays of P2X7 receptor activity can be readily performed by those skilled in the art.

As used herein, the term, “IC₅₀,” is a functional measure that refers to the molar concentration of a receptor antagonist that will diminish a downstream effect of the receptor by 50%. IC₅₀ is measured at a given concentration of receptor agonist, usually a concentration that achieves about 70% to 100% of the maximal activity of the receptor. IC₅₀ values for P2X7 receptor antagonists can be readily determined by those skilled in the art.

As used herein, the phrase, “effective concentration of extracellular ATP,” refers to the amount of extracellular (free) ATP available to activate the P2X7 receptor.

As used herein, the phrases, “enzyme that degrades ATP,” and, “ATP degrading enzyme,” are used interchangeably and refer to all enzymes that degrade ATP. The phrases, “enzyme that degrades ATP,” and, “ATP degrading enzyme,” also include enzymes that function to diminish levels of ATP by interconverting ATP and its degradation products. ATP degradation products include ADP, AMP, adenosine, inosine, and hypoxanthine. Enzymes that degrade ATP may be derived from any organism including humans. As used herein, the term, “ecto-ATPase,” refers to one or more ATP degrading enzymes that degrade extracellular ATP.

As used herein, the verb, “inhibit,” in reference to an enzyme means to diminish the activity of the enzyme by at least 5%, at least 10%, at least 15%, or more below a baseline or reference level of activity, such as, at a given concentration of reactant, as measured by a downstream product of the enzyme reaction.

As used herein for the enzymes NTPDase 1, NTPDase 2, and potato apyrase, “unit,” and, “U,” are synonymous, and one unit refers to an amount of enzyme that degrades one micromole of ATP per minute at 37° C. with a standardized assay that can approximate physiologic conditions. The determination of enzyme potency in units or in units/mg protein can be readily determined by those skilled in the art.

As used herein, the phrase, “NAD-mediated activation of the P2X7 receptor,” refers to activation of the P2X7 receptor through the pathway that utilizes NAD as a substrate for ADP-ribosylation of the P2X7 receptor.

As used herein, “topical administration,” refers to delivery of a therapeutic composition directly onto the surface of the cornea and conjunctiva. A composition delivered via topical administration can be applied, for example, as an eye drop or via a device such as, for example, a contact lens, collagen shield, or slow release reservoir.

As used herein, “active substance,” and, “active ingredient,” are used interchangeably and refer to the components of a composition that attenuate the activity of the P2X7 receptor. Such components include a P2X7 receptor inhibitor, an ATP degrading enzyme, a compound that diminishes the effective concentration of ATP, and a compound that diminishes NAD-mediated activation of the P2X7 receptor.

As used herein, “effective amount,” refers to a concentration of the active substance in a composition, which, when administered to an eye, will mitigate damage to the cornea and conjunctiva and/or treat a disorder of the cornea and conjunctiva.

As used herein, “ophthalmically acceptable,” with respect to a formulation, composition or ingredient herein means having no persistent detrimental effect on the treated eye or the functioning thereof, or on the general health of the subject being treated. It will be recognized that transient effects such as minor irritation or a stinging sensation do occur with topical ophthalmic administration of drugs and the existence of such transient effects is not inconsistent with the formulation, composition or ingredient in question being, “ophthalmically acceptable,” as herein defined. However, formulations, compositions, and ingredients that cause no substantial detrimental effect, even of a transient nature, can be beneficial.

As used herein, “pharmaceutically acceptable carrier,” refers to any vehicle that is ophthalmically acceptable and provides delivery of an effective amount of at least one active substance to the cornea and/or conjunctiva.

As used herein, the phrase, “gene therapy,” refers to a method of therapy in which cells of the eye and/or surrounding tissues are genetically modified such that the cells produce, in situ, one or more therapeutic entities that inhibit P2X7 receptor function, degrade ATP or diminish the effective concentration of ATP, and/or diminish NAD-mediated activation of the P2X7 receptor.

In some embodiments, the compositions of the present invention are administered to the eye to mitigate damage to the cornea and conjunctiva associated with one or more DOCC. The compositions of the present invention can be used to mitigate damage to the cornea and conjunctiva associated with one or more DOCC in any mammal. The compositions of the present invention can be used to mitigate damage to the cornea and conjunctiva associated with one or more DOCC in a human.

In one embodiment, the compositions of the present invention are administered to the eye to mitigate damage to the cornea and conjunctiva associated with one or more allergic disorders of the cornea and conjunctiva. In one embodiment, the compositions of the present invention are administered to the eye to mitigate damage to the cornea and conjunctiva associated with one or more corneal and conjunctival disorders related to injury. In one embodiment, the compositions of the present invention are administered to the eye to mitigate damage to the cornea and conjunctiva associated with one or more disorders associated with blepharitis. In one embodiment, the compositions of the present invention are administered to the eye to mitigate damage to the cornea and conjunctiva associated with one or more disorders related to dry eye. In one embodiment, the compositions of the present invention are administered to the eye to mitigate damage to the cornea and conjunctiva associated with one or more corneal and conjunctival disorders related to causes other than allergy, injury, blepharitis, and dry eye.

In some embodiments, the compositions of the present invention are administered to the eye to treat one or more DOCC. The compositions of the present invention can be used to treat one or more DOCC in any mammal. The compositions of the present invention can be used to treat one or more DOCC in a human.

In one embodiment, the compositions of the present invention are administered to the eye to treat one or more allergic disorders of the cornea and conjunctiva. In one embodiment, the compositions of the present invention are administered to the eye to treat one or more corneal and conjunctival disorders related to injury. In one embodiment, the compositions of the present invention are administered to the eye to treat one or more disorders associated with blepharitis. In one embodiment, the compositions of the present invention are administered to the eye to treat one or more disorders related to dry eye. In one embodiment, the compositions of the present invention are administered to the eye to treat one or more corneal and conjunctival disorders related to causes other than allergy, injury, blepharitis, and dry eye.

In one embodiment, the compositions of the present invention are administered to the eye to treat one or more symptoms and signs of uveitis or iritis including injected conjunctiva, redness of the eye, blurred vision, photophobia, floaters, eye pain, headaches, loss of peripheral vision, flashes of light, dilated ciliary vessels, presence of cells and flare in the anterior chamber, and keratic precipitates on the posterior surface of the cornea.

In some embodiments, the compositions of the present invention are used to manufacture a medicament to mitigate damage to the cornea and conjunctiva associated with one or more DOCC. In one embodiment, the compositions of the present invention are used to manufacture a medicament to mitigate damage to the cornea and conjunctiva associated with one or more allergic disorders of the cornea and conjunctiva. In one embodiment, the compositions of the present invention are used to manufacture a medicament to mitigate damage to the cornea and conjunctiva associated with one or more corneal and conjunctival disorders related to injury. In one embodiment, the compositions of the present invention are used to manufacture a medicament to mitigate damage to the cornea and conjunctiva associated with one or more disorders associated with blepharitis. In one embodiment, the compositions of the present invention are used to manufacture a medicament to mitigate damage to the cornea and conjunctiva associated with one or more disorders related to dry eye. In one embodiment, the compositions of the present invention are used to manufacture a medicament to mitigate damage to the cornea and conjunctiva associated with one or more corneal and conjunctival disorders related to causes other than allergy, injury, blepharitis, and dry eye.

In some embodiments, the compositions of the present invention are used to manufacture a medicament to treat one or more DOCC. In one embodiment, the compositions of the present invention are used to manufacture a medicament to treat one or more allergic disorders of the cornea and conjunctiva. In one embodiment, the compositions of the present invention are used to manufacture a medicament to treat one or more corneal and conjunctival disorders related to injury. In one embodiment, the compositions of the present invention are used to manufacture a medicament to treat one or more disorders associated with blepharitis. In one embodiment, the compositions of the present invention are used to manufacture a medicament to treat one or more disorders related to dry eye. In one embodiment, the compositions of the present invention are used to manufacture a medicament to treat one or more corneal and conjunctival disorders related to causes other than allergy, injury, blepharitis, and dry eye.

In one embodiment, the compositions of the present invention are used to manufacture a medicament to treat uveitis or iritis.

In one embodiment, compositions of the present invention are administered to the eye via topical administration to the cornea and conjunctiva. In another embodiment, compositions of the present invention can be administered to the eye via subconjunctival injection, via retrobulbar injection, via injection into Tenon's capsule, via injection into the anterior chamber of the eye, via injection into the posterior chamber of the eye, or via injection into the vitreous, or any combination thereof.

In one embodiment, compositions of the present invention comprise an inhibitor of the P2X7 receptor. In one embodiment, compositions of the present invention comprise a reversible inhibitor of the P2X7 receptor. In one embodiment, compositions of the present invention comprise an irreversible inhibitor of the P2X7 receptor.

Examples of P2X7 inhibitors include Coomassie Brilliant Blue G (BBG), periodate oxidized ATP (oxidized ATP or oxATP), pyridoxalphosphate-6-azophenyl-2′,4′-disulphonic acid (PPADS), pyridoxal-5′-phosphate-6-(2′-napthylazo-6′-nitro-4′,8′-disulphonate (PPNDS), suramin, calmidizolamide, KN-62, KN-04, A-438079, A-740003 (Journal of Pharmacology and Experimental Therapeutics 2006 319:1376), A-839977, AZ-10606120, AZ-11645373 (British Journal of Pharmacology 2006 149:880), AZ-10573295, AZ-11648720, AZD-9056, CE-224,535, EVT-401, GSK-1482160, GW-791343, and P18 (Journal of Biological Chemistry 2010 285:21165).

Examples of P2X7 inhibitors further include those described in the following publications and in the references cited therein, each of which is incorporated by reference in entirety in the present application: Recent Pat CNS Drug Discov 2010 5:35; Expert Opinion on Therapeutic Patents 2010 20:625; Journal of Medicinal Chemistry 2009 52:3123; Bioorg. Med. Chem. Lett. 2008 18:571; British Journal of Pharmacology 2007 151:571; British Journal of Pharmacology 2009 157:1203; British Journal of Pharmacology 2009 156:1312; Journal of Medicinal Chemistry 2006 49:3659; and Expert Opinion Investig Drugs 2011 20:897.

Examples of P2X7 inhibitors further include those described in the following publications of patent applications and U.S. Pat. Nos. 6,831,193; 7,935,832; 7,932,282; 7,718,693; 7,919,503; 7,235,657; 7,071,223; 6,974,812; WO9929660; WO9929661; WO9929686; WO2005014555; WO2005025571; WO2006110516; WO2007056046; US20060025614; WO2006086229; US20050171195; WO2008003697; WO2001094338; WO2000061569; WO2001044170; WO2001042194; WO2004074224; WO2006025783; WO2003041707; WO2003080579; WO2005014529; WO2006059945; WO2004106305; WO2006080884; WO2008013494; WO2005009968; WO2000071529; WO2004058270; WO2003042191; WO2006003513; WO2006003517; WO2004099146; WO2004058731; WO2007109172; WO2007109182; WO2007109160; WO2007109154; WO2007109201; WO2007109192; WO2006102588; WO2006102610; WO2007028022; WO2008066789; WO2008064432; WO2005019182; WO2007141267; WO2007141269; WO2005111003; WO2007056091; WO2001044213; WO2001046200; WO2008005368; WO2003047515; WO2003059353; WO2008114002; WO2009070116; WO2009019503; WO2008112205; US20090170838; WO2008124153; WO2009012482; WO2009023623; WO2008116814; WO2008116845; WO2008119685; WO2008119825; WO2008125600; WO2008138876; WO2009053459; WO2009077362; WO2009077559; WO2009108551; WO2009002423; WO2009057827; WO2009132000; FR2918568; US20090215727; WO2009118175; US20070142329; US20070281939; US20060217430; US20080039478; US20070225324; US20070197565; US20060217448; US20060276505; US20080146612; US20080132550; US20080009541; US20070259920; US20070105842; US20080076924; US20090312366; US20100160384; US20100160387; US20100160388; US20100267762; US20100286390; US20110028502; US20110071143; and US20110212992.

Examples of P2X7 inhibitors further include polyclonal and monoclonal antibodies, or antigen-binding portions thereof, that bind to and neutralize the function of the P2X7 receptor.

Examples of P2X7 inhibitors further include aptamers and spiegelmers that bind to and neutralize the function of the P2X7 receptor. An aptamer is an oligonucleotide or polypeptide molecule that binds to a specific target molecule. A spiegelmer is an L-enantiomer oligonucleotide that binds a specific target molecule.

Examples of P2X7 inhibitors further include siRNA moieties that, when delivered to cells, enter the cells and inhibit expression of the P2X7 receptor or interfere with trafficking of the P2X7 receptor to the cell surface.

Whereas the above examples of P2X7 receptor inhibitors are meant to impart an understanding of the scope of the present invention, the examples and listings are not intended to provide an exhaustive list of all P2X7 receptor inhibitors. It is recognized that those skilled in the art can identify a specific P2X7 receptor inhibitor that is not included in the above examples, but that would nonetheless constitute a P2X7 receptor inhibitor according to the present invention.

In one embodiment, the compositions of the present invention comprise a P2X7 receptor inhibitor that is a non-selective inhibitor; that is, one that inhibits activation of the P2X7 receptor, but also inhibits other P2 receptors. Non-specific P2X7 receptor inhibitors usually, but not always, display greater potency for inhibiting the P2X7 receptor as compared to other receptors.

In one embodiment, the P2X7 receptor inhibitor is BBG. It is known in the art that BBG has the capacity to bind to proteins via noncovalent interactions. According to the present invention, this capacity for protein binding, followed by slow dissociation, can create a slow release depot of BBG and thus provides a means of prolonging BBG bioavailability on the ocular surface after topical administration.

In another embodiment, the P2X7 receptor inhibitor is oxATP. Oxidized ATP is an irreversible inhibitor of the P2X7 receptor (Journal of Biological Chemistry 1993 268:8199). According to the present invention, irreversible inhibition provides a means of prolonging therapy after topical administration since the therapeutic effect will persist until new P2X7 receptors are generated by corneal and conjunctival cells as well as by infiltrating inflammatory cells. Additionally, oxATP has the capacity to inhibit other receptors and enzymes, which can provide a synergism with P2X7 receptor inhibition to potentiate its therapeutic effect (British Journal of Pharmacology 2003 140:441).

Oxidized ATP is a dialdehyde derivative of ATP that was described more than three decades ago as an affinity label for the ATP binding sites of various enzymes (European Journal of Biochemistry 1976 62:125, Biochem. Soc. Trans. 1979 7:239, Biochem. Soc. Trans. 1979 7:1131, Biochem. Soc. Trans. 1979 7:1133, Biochemistry 1982 21:4073, Bioorganic Chemistry 1982 11:55, and Biochimica et Biophysica Acta 1990 1037:216). Based on its resemblance to native ATP, oxATP is able to efficiently and specifically insert into ATP binding sites. Within a binding site, its reactive aldehyde groups can form a permanent covalent bond with the primary amine groups of nearby lysines through a series of chemical reactions initiated by the formation of a Schiff base. In 1993, oxATP was identified as a P2X7 inhibitor (Journal of Biological Chemistry 1993 268:8199; note, in 1993, P2X7 was referred to as P2Z). Since it covalently modifies, and thereby permanently inactivates the P2X7 receptor, oxATP is an irreversible P2X7 inhibitor. In 2003, oxATP was found to alter immune function through mechanisms that are unrelated to P2X7 blockade and that remain poorly understood (British Journal of Pharmacology 2003 140:441 and British Journal of Pharmacology 2003 140:507).

Oxidized ATP has also been shown to increase levels of extracellular ATP by attenuating its degradation. Specifically, oxATP has been shown to inhibit ecto-ATPase (Journal of Biological Chemistry 1993 268:8199 and British Journal of Pharmacology 2003 140:507). Thus, oxATP is a unique therapeutic in its potential to increase extracellular ATP, thereby preserving the beneficial aspects of ATP signaling, while blocking the P2X7 receptor-mediated detrimental effects of ATP signaling. Beneficial aspects of extracellular ATP signaling may include, for example, increased tear production.

According to the present invention, oxATP is distinguished from other P2X7 inhibitors by one or more of its attributes: the ability to irreversibly inactivate the P2X7 receptor; the ability to covalently modify multiple ATP binding proteins; the ability to alter immune function through mechanisms unrelated to P2X7 inhibition; and the ability to inhibit ecto-ATPase and increase levels of extracellular ATP. The chemistry of oxATP is reviewed in the Russian Journal of Bioorganic Chemistry 2000 26:429.

It is recognized that the pharmacologic and scientific literature is replete with chemical derivatives of ATP (for examples, see J. Cell. Sci. 2001 114:459 and the poster insert). Thus, one skilled in the art could chemically modify oxATP to alter its properties such as its potency or its stability. For example, strategies to improve stability include, but are not limited to, substituting the hydrogen on the 4′ carbon (with numbering analogous to the ATP ribose ring) with a methyl or hydroxyl group and substituting an oxygen on the alpha phosphate with a sulfur.

Accordingly, any chemical derivative of oxATP that preserves its mechanism of action, that is, specific entry into ATP binding sites followed by aldehyde-mediated covalent bond formation, would constitute an irreversible inhibitor of the P2X7 receptor of the present invention.

In one embodiment, the compositions of the present invention comprise a P2X7 receptor inhibitor that is a selective inhibitor, with minimal or no detectable effects on other P2 receptors.

An effective amount for a given P2X7 receptor inhibitor in a composition of the present invention will usually, but not always, be a concentration that is 100 fold below to 100 fold above the IC₅₀ for that inhibitor, 50 fold below to 50 fold above the IC₅₀ for that inhibitor, 10 fold below to 10 fold above the IC₅₀ for that inhibitor, 5 fold below to 5 fold above the IC₅₀ for that inhibitor.

In some embodiments, compositions of the present invention comprise a compound that decreases the effective concentration of extracellular ATP. In one embodiment, the compound that decreases the effective concentration of extracellular ATP is an enzyme that degrades ATP.

Examples of enzymes that degrade ATP include NTPDase 1, NTPDase 2, NTPDase 3, NTPDase 8, potato apyrase, ecto-nucleotide pyrophosphatase phosphodiesterases 1 through 7 (E-NPPs), alkaline phosphatases, dinucleoside polyphosphate hydrolases, adenylate kinase, nucleoside diphosphate kinase, and ecto-F1-Fo ATP synthase. Additional examples of enzymes that degrade ATP, but with higher affinity for other nucleotides, include NTPDases 4 through 7.

Examples of enzymes that degrade ATP are described in publications, Purinergic Signaling 2006 2:409, Purinergic Signaling 2011 7:21, Biochimica and Biophysica Acta 2008 1783:673, Purinergic Signaling 2006 2:351, and U.S. Pat. No. 7,264,809.

Whereas the above examples of enzymes that degrade ATP are meant to impart an understanding of the scope of the present invention, the examples and listings are not intended to provide an exhaustive list of all enzymes that degrade ATP. It is recognized that those skilled in the art can identify a specific enzyme that degrades ATP that is not included in the above examples, but that would nonetheless constitute an enzyme that degrades ATP according to the present invention.

In one embodiment of the present invention, the enzyme that degrades ATP is a soluble form of NTPDase 1 or NTPDase 2. NTPDase 1 degrades ATP to yield mostly AMP. NTPDase 2 degrades ATP to yield mostly ADP. An effective amount of NTPDase 1 or NTPDase 2 in a composition of the present invention can, but not always, be 0.001 units/ml to 1,000 units/ml, 0.01 units/ml to 100 units/ml, 0.1 units/ml to 10 units/ml. A soluble form of human NTPDase 1 is described in U.S. Pat. No. 7,264,809 and the Journal of Clinical Investigation 1998 101:1851. A soluble form of NTPDase 2 can be generated via the same strategy as described for NTPDase 1.

In some embodiments, compositions of the present invention comprise enzymes that further degrade the degradation products of ATP. In one embodiment, compositions comprise the enzyme 5′ ecto-nucleotidase which converts AMP into adenosine. In an embodiment, the 5′ ecto-nucleotidase is a soluble form of 5′ ecto-nucleotidase, which, for example, is described in Molecular Pain 2010 6:20. In another embodiment, compositions of the present invention comprise adenosine deaminase, which converts adenosine into inosine. In another embodiment, compositions of the present invention comprise purine nucleoside phosphorylase, which converts inosine to hypoxanthine.

In one embodiment, compositions of the present invention comprise a compound, other than an enzyme that degrades ATP, which decreases the effective concentration of extracellular ATP. Such compounds include molecules that block the release of ATP from cells and compounds that prevent extracellular ATP from engaging the P2X7 receptor, such as those that entrap or sequester ATP. Molecules that can block the release of ATP from cells include Cl⁻ channel blockers and hemichannel blockers. Examples of Cl⁻ channel blockers include: 5-nitro-2-(3-phenylpropyl-amino)-benzoic acid (NPPB); 4-acetamido-4′-isothiocyanostilbene-2,2′-disulphonic acid (SITS); niflumic acid; 4,4′-diisothiocyanatostilbene-2,2′-disulphonic acid (DIDS); anthracene-9-carboxylic acid (A9C); N-phenylanthranilic acid; diphenylamine-2-carboxylic acid (DPC); R(+)methylindazone, indanyloxyacetic acid 94 (IAA-94); 2-aminomethyl phenols; and 2-aminomethyl-4-(1,1′-dimethyl ethyl)-6-iodophenol hydrochloride (2) disulphonic stilbenes (MK-447). Examples of hemichannel blockers include: mefloquine acid; meclofenamic acid; retinoic acid; 18-α-glycyrrhetinic acid; flufenamic acid; niflumic acid; carbenoxolone; and connexin mimetic peptides. Compounds that prevent extracellular ATP from engaging the P2X7 receptor, to dampen signaling induced thereby, include molecules that bind or sequester ATP such as: polyclonal and monoclonal antibodies, or the antigen-binding portions thereof, that bind to ATP; aptamers and spiegelmers that bind ATP; other ligands that bind ATP, such as, the extracellular portions or domains of purinergic receptors; and so on. Compounds that entrap or sequester ATP can be derived by those skilled in the art from many of the ATP binding proteins that have been described in the art.

Whereas the above examples of compounds that decrease the effective concentration of extracellular ATP are meant to impart an understanding of the scope of the present invention, the examples and listings are not intended to provide an exhaustive list of all compounds that decrease the effective concentration of extracellular ATP. It is recognized that those skilled in the art can identify a specific compound that is not included in the above examples, but that would nonetheless constitute a compound that decreases the effective concentration of extracellular ATP according to the present invention.

In some embodiments, the present invention comprises compositions that inhibit NAD-mediated activation of the P2X7 receptor. Extracellular NAD serves as a substrate for enzymes of the ecto-ADP-ribosyltransferase (ART) family, which transfer ADP-ribose groups to the P2X7 receptor, and, in doing so, activate the receptor. This process is opposed by enzymes of the NAD-glycohydrolase family, including CD38, which hydrolyze and diminish the extracellular concentration of NAD. Additionally, enzymes have been described that have the potential to deactivate the receptor by removing the ADP-ribose moieties. The role of ADP-ribosylation in regulating receptor function is reviewed in Current Medicinal Chemistry 2004 11:857.

NAD-mediated activation of the P2X7 receptor can be inhibited with known P2X7 receptor inhibitors, for example, oxATP and KN-62. However, NAD-mediated activation of the P2X7 receptor can also be inhibited with compounds specific to the ADP-ribosylation pathway.

Examples of compounds that inhibit NAD-mediated activation of the P2X7 receptor include: 1,N⁶-etheno NAD (eNAD); nicotinamide hypoxanthine-dinucleotide (NHD); and nicotinamide guanine-dinucleotide (NGD). These compounds are derived from NAD but have a modified adenine group such that P2X7 receptor ribosylation does not lead to activation (Current Medicinal Chemistry 2004 11:857).

Examples of compounds that inhibit NAD-mediated activation of the P2X7 receptor also include compounds that inhibit ART function: agmatine; arginine methyl ester; m-Iodobenzylguanidine; diethylamino(benzylideneamino) guanidine (DEA-BAG); niacin; 3-aminobenzamide; vitamins K1 and K3; 1,8-naphthalamide; novobiocin; coumermycin A1; and unsaturated fatty acids (Current Medicinal Chemistry 2004 11:857). Additional examples of compounds that inhibit ART function include: polyclonal or monoclonal antibodies, or antigen-binding portions thereof, that bind and neutralize ART; aptamers and spiegelmers that bind and neutralize ART; and siRNA moieties that attenuate ART expression.

An alternative means of inhibiting NAD-mediated activation of the P2X7 receptor includes increasing the activity of an NAD-glycohydrolase, for example, with a composition comprising soluble CD38 protein.

Whereas the above examples of compounds that inhibit NAD-mediated P2X7 receptor activation are meant to impart an understanding of the scope of the present invention, the examples and listings are not intended to provide an exhaustive list of all compounds that inhibit NAD-mediated P2X7 receptor activation. It is recognized that those skilled in the art can identify a specific compound that is not included in the above examples, but that would nonetheless constitute a compound that inhibits NAD-mediated P2X7 receptor activation according to the present invention.

Compositions of the present invention can comprise one or more active substances. That is, the compositions can comprise one or more P2X7 inhibitors, one or more enzymes that degrade ATP or compounds that diminish the effective concentration of ATP, or one or more compounds that that diminish NAD-mediated activation of the P2X7 receptor, or any combination thereof.

The compositions of the present invention can be used as single agents or in combination with other ocular medications. The compositions of the present invention can be combined with known drugs for treating ocular diseases into a single composition. Examples of such drugs include, but are not limited to, antihistamines, mast cell stabilizers (e.g. sodium cromoglycate), nonsteroidal anti-inflammatory agents, corticosteroids, immunomodulatory agents (e.g. cyclosporine, tacrolimus, mycophenolate mofetil, leflunomide, rapamycin, laquinimod, and infliximab), antibiotics, antiviral agents, and pain medications.

The dosing schedules for compositions of the present invention will usually, but not always, be no more frequent than a topical administration every two hours and no less frequent than a topical administration once per week. The compositions can be administered topically to the eye one to four times per day.

The effective amount of an active substance in conjunction with a dosing schedule to treat specific DOCC will be readily ascertainable by those skilled in the art through animal modeling and clinical trials.

The present invention also discloses gene therapy methods for inhibiting P2X7 receptor function, diminishing the effective concentration of extracellular ATP, and/or inhibiting NAD-mediated activation of the P2X7 receptor. The gene therapy methods of the present invention can be used to mitigate damage to the cornea and conjunctiva associated with one or more DOCC, to treat one or more DOCC, or both in any mammal. The gene therapy methods of the present invention can be used to mitigate damage to the cornea and conjunctiva associated with one or more DOCC, to treat one or more DOCC, or both in a human.

In some embodiments, compositions of the present invention comprise one or more gene transfer vectors. In one embodiment, the compositions comprising one or more gene transfer vectors are administered to the eye via topical administration to the cornea and conjunctiva. In another embodiment, the compositions comprising one or more gene transfer vectors are administered, for example, via subconjunctival injection, via retrobulbar injection, via injection into Tenon's capsule, via injection into the anterior chamber of the eye, via injection into the posterior chamber of the eye, via injection into the vitreous, or via any combination thereof.

Gene transfer vectors encode recombinant DNA and RNA molecules and are capable of delivering this genetic material to cells of the eye and/or surrounding tissues including cells of the cornea and conjunctiva. The genetically-modified cells express the encoded recombinant DNA and RNA molecules to generate therapeutic entities in situ. Examples of vectors include those derived from viruses such as adenoviral vectors, retroviral vectors, lentiviral vectors, adeno-associated viral vectors (AAV), and the like. Additional examples of vectors include synthetic (also called non-viral) vectors such as “naked” DNA molecules and DNA and/or RNA molecules complexed with cationic liposomes, anionic liposomes, dendrimers, polyethyleneimine, antibody or peptide targeting molecules, nanoparticles, and the like. According to the present invention, compositions comprising gene transfer vectors can be administered in conjunction with techniques that enhance gene transfer including ballistic gene delivery, ultrasound, electroporation, iontophoresis, and femtosecond laser exposure. Corneal gene therapy is reviewed in Clinical and Experimental Ophthalmology 2010 38:93.

In some embodiments, vector-delivered recombinant DNA and RNA molecules encode therapeutic entities that block P2X7 receptor function. In one embodiment, the recombinant DNA and RNA molecules encode a dominant negative form of the P2X7 receptor that inhibits the function of the native P2X7 receptor. Such dominant negatives can be generated experimentally or may exist as naturally occurring P2X7 receptor variants (Molecular Pharmacology 2004 65:646, Journal of Biological Chemistry 2006 281:17228). In one embodiment, the recombinant DNA and RNA molecules encode a protein such as an antibody, or the antigen-binding portions thereof, which can bind and inhibit the P2X7 receptor. In one embodiment, the recombinant DNA and RNA molecules encode an RNA moiety such as a ribozyme, antisense RNA, or siRNA molecule that blocks the ability of the genetically-modified cells to produce P2X7 receptors.

In one embodiment, the vector-delivered recombinant DNA and RNA molecules encode a therapeutic protein such as an enzyme that degrades ATP, a 5′-nucleotidase, and the like. Such therapeutic proteins can be expressed in the genetically-modified cells as membrane bound proteins or they can be secreted as soluble proteins to enable diffusion throughout the cornea and conjunctiva. In one embodiment, the vector-delivered recombinant DNA and RNA molecules encode an enzyme that inhibits NAD-mediated P2X7 receptor activation, for example, an NAD-glycohydrolase, such as CD38, or a dominant negative ecto-ADP-ribosyltransferase.

Gene transfer vectors also encode regulatory elements, such as promoters, which control the expression of the encoded recombinant DNA and RNA molecules. In one embodiment, the gene transfer vector encodes a promoter that is a constitutive promoter; that is, one which continuously expresses the therapeutic entity. Examples of constitutive promoters include the CMV and PGK promoters. In another embodiment, the gene transfer vector encodes a conditional promoter; that is, one that can be activated or inactivated by cellular functions such as, for example, by P2X7 receptor activation. In this case, the conditional promoter can be engineered to encode regulatory elements from the promoters of genes whose transcription is regulated by the P2X7 receptor. In one embodiment, the gene transfer vector encodes a promoter that is activated or silenced by an exogenously supplied small molecule such as, for example, tetracycline, ecdysone, mifepristone, tamoxifen, and rapamycin. The regulation of vector gene expression is reviewed in Trends in Molecular Medicine 2008 14:410.

The dosing schedule for the gene transfer vectors of the present invention will be readily ascertainable by those skilled in the art through animal modeling and clinical trials and will usually, but not always, be one administration annually, monthly, weekly, daily.

Whereas the above examples of gene therapy methods to mitigate damage to the cornea and conjunctiva and to treat DOCC are meant to impart an understanding of the scope of the present invention, the examples and listings are not intended to provide an exhaustive list of all such gene therapy methods. It is recognized that those skilled in the art can identify a specific gene therapy method that is not included in the above examples, but that would nonetheless constitute a method of utilizing gene therapy to mitigate damage to the cornea and conjunctiva and to treat DOCC according to the present invention.

Formulations and devices for drug delivery to the surface of the eye are well known to those skilled in the art and are compatible with the surface of the eye. Such formulations and devices for drug delivery to the surface of the eye can also promote the stability and bioavailability of the therapeutic entity. Compositions of the present invention can comprise an aqueous solution, oil-based solution, cream, or ointment. Compositions of the present invention can be administered to the eye via an eye drop. Compositions of the present invention can be delivered to the eye via a drug delivery depot that functions as a reservoir to slowly release the active ingredient. Said drug delivery depot can comprise a solid matrix from which the active ingredient slowly disperses or a solid matrix that slowly dissolves or degrades thereby releasing the active ingredient. Said drug delivery depot can comprise an in situ-formed gel, a collagen corneal shield, a drug delivery device, or a contact lens.

The composition may be applied in ophthalmologic dosage forms known to those skilled in the art, such as preformed or in situ-formed gels or liposomes, for example, as disclosed in U.S. Pat. No. 5,718,922.

In another embodiment, the composition may be delivered via a contact lens or other object temporarily resident on the surface of the eye. For example, U.S. Pat. No. 6,410,045 describes a contact lens-type drug delivery device comprising a polymeric hydrogel contact lens containing a drug substance. In other embodiments, supports such as a collagen corneal shield can be employed.

U.S. Pat. No. 4,014,335 describes an ocular drug delivery device placed in the cul-de-sac between the sclera and lower eyelid for administering the drug and acting as a reservoir. The drug can diffuse from the reservoir through at least one of the polymeric layers of the laminate.

In some embodiments, an emulsion-based formulation as disclosed in U.S. Pat. Nos. 5,578,586; 5,371,108; 5,294,607; 5,278,151; and 4,914,088 can be used, which reduces evaporation of the aqueous layer from the surface of the eye. Generally, an admixture of a charged phospholipid and a non-polar oil are applied to the eye surface, such as, in the form of a finely divided oil-in-water emulsion. Another approach is described in U.S. Pat. Nos. 4,818,537 and 4,804,539, where liposome compositions in the form of emulsions are provided.

Hence, essentially, the ophthalmic compositions of the present invention can be delivered by any of a variety of known administration means, a contact lens wash or storage medium and so on. Hydrophobicity of a compound of interest can be controlled by, for example, chemically modifying the compound, such as, using an ester thereof, conjugating with a more hydrophilic carrier compound, containing the compound with a formed structure such as a liposome, combining the compound with a surfactant or amphiphilic compound, and so on. Thus, an aqueous solution for use as a wash or drop, for example, can be a solution or a suspension, see U.S. Pat. No. 6,551,584, for example.

A container for the ophthalmic composition of the present invention is not particularly limited so long as usable for the particular form of the composition. Hence, pliable or flexible plastic or metal-based tubes can be used for creams or ointments and aqueous formulations can be contained in glass or plastic containers as known in the art. The container can include a separate eye dropper with a flexible bulb to enable the delivery of a single drop or multiple drops to the eye. The container can be configured to comprise a nozzle to enable the delivery of a single drop or multiple drops to the eye. Containers can be designed for multiple use or single use applications. The container can be designed to maintain the active ingredient separate from the pharmaceutically acceptable carrier such that the two are mixed at or around the time of use. Such a container could be used, for example, to improve the stability and shelf-life of the active ingredient. Examples of plastics include polyethylene terephthalate, polyethylene, polypropylene and the like.

One skilled in the art may recommend a dosage schedule and dosage amount adequate for the subject being treated. For topical delivery, dosing may occur one to four times daily for as long as needed. The dosing may occur less frequently if the compositions are formulated in sustained delivery vehicles, are delivered via devices such as contact lenses or slow release reservoirs, or are delivered via gene transfer vectors. In the case of a drop, the dosage amount may be one or two drops per dose. The dosing schedule may also vary depending on the concentration of the active substance, which may depend on the actual compounds used and on the needs of the patient. The concentration of the active substance can be in the range of about 0.001 μM to about 10 mM in the tissues and fluids, from 1 μM to about 5 mM, from about 10 μM to about 2.5 mM, from about 100 μM to about 1 mM for each active substance. The concentrations of the components of the composition can be adjusted by typical pharmacokinetic and dilution calculations to achieve such local concentrations. Alternatively, penetration of cornea and absorption into other tissues in the interior of the eye can be demonstrated using radiolabeled active agent.

What constitutes an effective amount for treatment and/or prophylaxis depends, among other factors, on: the particular compound or compounds being administered; the residence time on the eye provided by the particular formulation of the active substance; the species, age and body weight of the subject; the particular ophthalmic condition for which treatment or prophylaxis is sought; and/or the severity of the condition, for example.

Hence, as known in the art, the compositions of the instant invention can comprise a wide range of components as known in the art. Reference also can be made to U.S. Pat. Nos. 6,013,271; 6,267,985; 4,992,478; 5,645,854; 5,811,111; and 5,851,543. Examples of functional classes of ingredients are antifoaming agents, antimicrobial agents, antioxidants, binders, biological additives, bulking agents, chemical additives, colorants, denaturants, dispersants, deodorants, lubricants, analgesics, fragrances, humectants, thickeners, opacifying agents, plasticizers, preservatives, such as dichlorobenzyl alcohol, benzoic acid, methylparaben and phenyl, propellants, reducing agents, suspending agents (nonsurfactant), gelling agents, such as agar, petrolatum and mineral wax, ultraviolet light absorbers, and viscosity increasing agents (aqueous and non-aqueous).

A composition of the invention is conveniently but not necessarily formulated as an in situ gellable aqueous liquid, or can be administered as an ointment, cream or a drop. Typically each drop, generated by a conventional dispensing means, has a volume of about 10 to about 40 μl. From 1 to about 6 such drops typically provides a suitable dose of the active agent(s) of interest. Where the composition is administered in a form other than eye drops, for example, as an ointment or cream, an equivalent dose is provided. Such a dose can be administered as needed, for example, application 1 to about 6 times per day, from about 2 to about 4 times a day.

The ophthalmic solution of the present invention can contain various ophthalmically acceptable additives, carriers, excipients, or diluents as appropriate, such as buffer, electrolytes, isotonicity agent, preservative, solubilizing agent, stabilizer, chelating agent, thickener, pH adjusting agent, and the like.

As the buffer, for example, boric acid or a salt thereof (sodium borate, etc.), citric acid or a salt thereof (sodium citrate, etc.), tartaric acid or a salt thereof (sodium tartrate, etc.), gluconic acid or a salt thereof (sodium gluconate, etc.), acetic acid or a salt thereof (sodium acetate, etc.), lactic acid or a salt thereof (sodium lactate, etc.), phosphoric acid or a salt thereof (sodium or potassium hydrogen phosphate, sodium or potassium dihydrogen phosphate, etc.), various amino acids such as glutamic acid, histidine, lysine, arginine, epsilon-aminocaproic acid and the like, and tris buffer, etc. can be mentioned. They can be used in a combination of one or more kinds thereof.

As electrolytes, for example, sodium chloride, potassium chloride, calcium chloride, magnesium chloride, sodium bicarbonate, and the like can be used to obtain compatibility with the surface of the eye.

As the isotonicity agent, for example, sorbitol, glucose, mannitol, glycerol, propylene glycol, glycerin, sodium chloride, potassium chloride, and the like can be mentioned.

As the preservative, for example, paraoxybenzoates, benzalkonium chloride, thimerosal, phenethyl alcohol, methyl paraben, propyl paraben, benzethonium chloride, benzyl alcohol, sorbic acid or a salt thereof, chlorhexidine or a salt thereof, sodium dehydroacetate, cetylpyridinium chloride, alkyldiaminoethylglycine hydrochloride, chlorobutanol and the like can be mentioned. They can be used in a combination of one or more kinds thereof.

As the solubilizing agent, for example, nonionic surfactants such as sorbitan polyoxyethylene fatty acid esters (polysorbate 80 and the like), polyoxyethylene hydrogenated castor oil, polyoxyethylene monostearate (polyoxyl stearate 40 and the like) and like, water-soluble polymers such as polyethylene glycol (macrogol 4000 and the like), poloxamers and the like, polyvinylpyrrolidone and the like, propylene glycol, and cyclodextrins and the like can be mentioned.

As the stabilizer, for example, disodium edetate, thiosodium sulfate, ascorbic acid, proline, cyclodextrins, condensed phosphoric acid or a salt thereof, sulfite, citric acid or a salt thereof, dibutylhydroxytoluene and the like can be mentioned. The stabilizer can include, for example, a conventionally known one such as hydroxypropylmethylcellulose, polyvinyl alcohol, carboxymethylcellulose, hydroxymethylcellulose, and glycerin. The stabilizer can include, for example, an oxidant. The stabilizer also can include, for example, a protein such as human serum albumin or gelatin.

The solubilizer includes, for example, a conventionally known one such as polyoxyethylene glycol ethers (e.g. sodium carboxymethylcellulose, polyoxyethylene lauryl ether, polyoxyethylene oleyl ether, etc.), polyethylene glycol higher fatty acid esters (e.g. polyethylene glycol monolaurate, polyethylene glycol monooleate, etc.) and polyoxyethylene fatty acid esters (e.g. polyoxyethylene sorbitan monolaurate, polyoxyethylene sorbitan monooleate, etc.).

The antioxidant includes, for example, ascorbic acid, sodium bisulfite, sodium thiosulfate, and thiolated compounds such as dithiothreitol, mercaptoethanol, acetylcysteine, glutathione, and the like.

The antiseptic includes, for example, a conventionally known one such as chlorobutanol, benzalkonium chloride, and cetylpyridinium chloride.

As the chelating agent, for example, sodium edetate, sodium citrate, condensed phosphoric acid or a salt thereof (condensed sodium phosphate etc.) and the like can be mentioned.

As the thickener, for example, methylcellulose, hydroxyethylcellulose, hydroxypropylcellulose, hydroxypropylmethylcellulose, sodium carboxymethylcellulose, polyvinylpyrrolidone, polyvinyl alcohol, polyethylene glycol, hyaluronic acid, and the like can be mentioned.

As the pH adjusting agent, for example, sodium hydroxide, potassium hydroxide, sodium carbonate, sodium hydrogencarbonate, boric acid or a salt thereof (sodium borate), hydrochloric acid, citric acid or a salt thereof (sodium citrate, sodium dihydrogen citrate etc.), phosphoric acid or a salt thereof (sodium dihydrogen phosphate, potassium dihydrogen phosphate etc.), acetic acid or a salt thereof (sodium acetate, ammonium acetate etc.), tartaric acid or a salt thereof (sodium tartrate etc.) and the like can be mentioned.

The ophthalmic formulation of the present invention can be prepared to have a pH from about 3 to about 10, from about 4 to about 8, from about 5 to about 7, from about 3.5 to about 6, from about 4 to about 6, from about 4 to about 5.5, from about 4 to about 5, from about 3 to about 5.

The ophthalmic solution of the present invention may contain, insofar as the objects of the invention are not impaired, one or more additional active agents to complement or to add to the medicinal impact of a composition of interest, for example, an ocular muscle modulator (neostigmine methylsulfate and the like), an astringent (zinc sulfate and the like), a drug, a vitamin (panthenol and the like), an amino acid, an anti-oxidant, hyaluronic acid, and the like.

The aqueous preparations can be made as known in the art; that is, hydrophilic compounds can be dissolved in a water or an aqueous buffer and the preparation sterilized, for example, by filtration, and then aliquoted.

The ophthalmically acceptable additives can include hydrophilic, amphipathic, and hydrophobic compounds to adjust the hydrophilicity and hydrophobicity of the formulation.

Ointments generally are substantially lipophilic and creams generally comprise an emulsion of both lipophilic and hydrophilic ingredients. An artisan would well recognize how to formulate such preparations to include the active agents as described herein.

Lipids for making an ointment or a cream can comprise an oil derived from animals, plants, nuts, petroleum etc. Those derived from animals, plant seeds, and nuts are similar to fats and consequently can contain one or a significant number of one or more polar acids and/or ester groups. Alternatively, oils derived from petroleum are usually aliphatic or aromatic hydrocarbons that are essentially free of polar groups. Other oil-based products that can be used include hydrocarbons or mineral fats obtained by the distillation of petroleum (petroleum jelly); vegetable oils and liquid triglycerides; animal fats or solid natural triglycerides; and waxes or solid ethers of fatty acids and organic alcohols. Lanolin or wool fats that are obtained from sheep wool and made up of fatty acids and cholesterol esters; and cetyl and stearyl alcohols, which are solid alcohols obtained by hydrogenation of their respective acids are also useable. Amphiphilic compounds such as soaps or salts of fatty acids, that may be acidic or basic depending on whether the hydrophilic group is anionic or cationic, sulfated alcohols which are semi-synthetic substances and synthetic surface active agents are known in the art and can be used in a preparation of interest for the intended use, for example, as a dispersing agent. Glycerin has hydrophilic properties and hence is useful as a humectant in a preparation of interest.

Other materials that may be used in a topical preparation of interest include liquid alcohols, liquid glycols, liquid polyalkylene glycols, liquid esters, liquid amides, liquid protein hydrolysates, liquid alkylated protein hydrolysates, liquid lanolin and lanolin derivatives and other like materials. Particular examples include monohydric and polyhydric alcohols, e.g., ethanol, isopropanol, glycerol, sorbitol, mannitol, cetyl alcohol and propylene glycol; ethers; polyethylene glycols and methoxypolyoxyethylenes; carbowaxes having molecular weights ranging from 200 to 20,000; polyoxyethylene glycerols; polyoxyethylene; sorbitols; stearoyl diacetin and so on.

A number of different emulsifiers or surfactants can be used to prepare an ointment of a cream of interest. Emulsifiers can be ionic or non-ionic. Examples of amphoteric surfactants and anionic surfactants useful in the compositions of the present invention include those disclosed in McCutcheon's, “Detergents and Emulsifiers”, North American edition (1986) and McCutcheon's, “Functional Materials”, North American Edition (1992); both of which are incorporated by reference herein in entirety. Surfactants that can be used include the betaines, sultaines and hydroxysultaines. Examples of other amphoteric surfactants are alkyliminoacetates, iminodialkanoates and aminoalkanoates.

An ointment can be comprised substantially of an oil, which is substantially solid or solidified at room temperature but with a melting temperature at or about body temperature, and which is ophthalmically acceptable. That lipoidal vehicle can comprise a petrolatum or a paraffin. A mineral oil can be included to attain a flowable consistency, for example. Also some water, lanolin and so on can be included, see U.S. Pat. No. 5,470,881, for example.

A cream comprises an emulsion of lipophilic and hydrophilic components, which are mixed into stable forms. Hence, lipophilic components are mixed to form a solution, and hydrophilic components are mixed to form a solution. Then the two solutions are mixed, one into the other as a design choice, to obtain a stable emulsion. Generally, creams containing more water can be thinner and may disperse more readily at warmer temperatures.

Compositions of the present invention may be stored frozen at, for example, −20° C., refrigerated at, for example, 4° C., or at ambient temperature, at, for example, 20° C.

A composition of interest is administered to a patient in need of treatment, namely, one with a disorder of the cornea and/or conjunctiva, at a desired dosing and course as determined empirically and as known in the art.

One specific aim of the present application is to provide a composition for application to an eye comprising an irreversible P2X7 inhibitor, oxATP, or a chemical derivative of oxATP that is an irreversible P2X7 inhibitor, and an ophthalmically acceptable formulation. Another specific aim of the present application is for said composition to comprise an aqueous solution, cream, or ointment. Another specific aim of the present application is for said composition to further comprise a container with a nozzle or eye dropper. One specific aim of the present application is for the composition comprising an irreversible P2X7 inhibitor, oxATP, or a chemical derivative of oxATP that is an irreversible P2X7 inhibitor, and an ophthalmically acceptable formulation to further comprise a drug delivery depot. Another specific aim of the present application is for that drug delivery depot to comprise an in situ-formed gel, a collagen corneal shield, a drug delivery device, or a contact lens. A specific aim of the present application is to provide for said irreversible P2X7 inhibitor of any of these compositions to also be an inhibitor of ecto-ATPase.

One specific aim of the present application is that the compositions of the aforementioned paragraph be used to treat an eye disease. Another specific aim of the present application is that said eye disease be a disorder of the cornea and conjunctiva (DOCC). Another specific aim of the present application is that said DOCC be dry eye or uveitis.

One specific aim of the present application is that the compositions for application to an eye comprising an irreversible P2X7 inhibitor, oxATP, or a chemical derivative of oxATP that is an irreversible P2X7 inhibitor, and an ophthalmically acceptable formulation, and which can further comprise an aqueous solution, cream, or ointment, and which can also be an inhibitor of ecto-ATPase be used to manufacture a medicament to treat an eye disease. Another specific aim of the present application is for said eye disease to be a DOCC. Another specific aim is for said DOCC to be dry eye or uveitis. Another specific aim of the present application is for said medicament to be packaged in a container with a nozzle or eye dropper. Another specific aim of the present application is for said medicament to further comprise a drug delivery depot. Another specific aim is for said drug delivery depot to comprise an in situ-formed gel, a collagen corneal shield, a drug delivery device, or a contact lens.

One specific aim of the present application is to provide a method for treating an eye disease comprising topical administration of the compositions comprising an irreversible P2X7 inhibitor, oxATP, or a chemical derivative of oxATP that is an irreversible P2X7 inhibitor, and an ophthalmically acceptable formulation, and which can further comprise an aqueous solution, cream, or ointment, and which can also be an inhibitor of ecto-ATPase to an eye comprising said eye disease. Another specific aim of the present application is for said eye disease to be a DOCC. Another specific aim is for said DOCC to be dry eye or uveitis.

One specific aim of the present application is to provide a composition for application to an eye comprising a reversible P2X7 inhibitor, and an ophthalmically acceptable formulation packaged in a container with a nozzle or eye dropper. Another specific aim of the present application is for said composition to be used to treat a DOCC.

The invention now will be exemplified in the following non-limiting examples.

### EXAMPLES

### Example 1

### Commercially Available Ophthalmic Formulation

Compositions of the present invention can be formulated in aqueous or oil based vehicles that are well known to those skilled in the art and are currently used for commercially available products. One formulation for the compositions of the present invention is BSS Sterile Irrigating Solution (Alcon Laboratories, Fort Worth, Tex.). The components include sodium chloride 0.64%, potassium chloride 0.075%, calcium chloride 0.048%, magnesium chloride 0.03%, sodium acetate 0.39%, sodium citrate 0.17%, sodium hydroxide and/or hydrochloric acid to adjust the pH to 7.4, and water. For the present invention, carboxymethylcellulose (CMC) (0.5-1.5%), or other known equivalent thickeners, such as hydroxypropyl methyl cellulose (HPMC) can be added to increase dwell time on the eye.

### Example 2

### Table of Commercially Available Lubricating Ophthalmic Compositions

Compositions of the present invention can be prepared with formulations similar or identical to the lubricating compositions currently available for dry eye. Such formulations are designed to have extended dwell times on the ocular surface and are thus well-suited for delivery of the active substances of the present invention. Table 1 provides an overview of the currently available lubricating compositions with a brief description of each.

TABLE 1
Commercially Available Lubricating Eye Drops
Artificial Tears
Carboxymethylcellulose (CMC) Artificial Tears

| | |
|---|---|
| Optive | Carboxymethylcellulose sodium 0.5%, Purite preserved. |
| | contains compatible solutes (glycerin 0.9%, erythritol, |
| | levocarnitine), which protect against cell damage by |
| | hyperosmolar tears (osmoprotection). |
| Refresh Tears | 0.5% CMC Purite as preservative. Refresh Plus Tears also has |
| | 0.5% CMC, but is preservative-free & comes in single use |
| | vials. |
| Refresh Liguigel | 1% CMC Purite as preservative. Refresh Celluvisc also has 1% |
| | CMC, but is preservative-free & comes in single use vials. |
| Thera Tears | 0.25% CMC, hypotonic, preservative-free. |

Polyethylene Glycol Artificial Tears

| | |
|---|---|
| Systane | Polyethylene glycol 400 0.4% (lubricant), propylene glycol |
| | 0.3% (lubricant), hydroxypropyl (HP) guar (gel-forming |
| | matrix) Polyquaternium-1 as preservative. |
| Systane Ultra | This is an improvement on original Systane. The delivery |
| | system ensures minimal blurring of vision. In the bottle, |
| | Systane Ultra consists of a loosely cross-linked meshwork |
| | created by interactions between borate and HP-guar, which is |
| | kept loose due to the presence of sorbitol. Upon instillation HP- |
| | guar forms a stronger, tighter viscoelastic gel that covers and |
| | lubricates damaged corneal cells. |
| Blink Tears | Polyethylene glycol 400 0.25% (lubricant), sodium chlorite |
| | (preservative) has hyaluronic acid (HA), which promotes |
| | healing of the corneal epithelium. |

Hydroxypropyl Mehylcellulose (HPMC) Artificial Tears

| | |
|---|---|
| Bion Tears | 0.3% HPMC, 0.1% dextran 70. (bicarbonate & zinc help mucus |
| | & surface cells), preservative-free. |
| Tears Naturale Forte | 0.3% HPMC, 0.1% dextran 70, 0.2% glycerin, Polyquad |
| | 0.001% as preservative. |
| Tears Natural II | 0.3% HPMC, 0.1% dextran 70, Polyquad 0.001% as |
| | preservative. |
| Tears Natural Free | 0.3% HPMC, 0.1% dextran 70, preservative-free. |
| Genteal | 0.3% HPMC, GenAqua (sodium perborate) as preservative. |
| | Genteal is also available as preservative-free single-use vials. |
| Genteal Mild | 0.2% HPMC, GenAqua (sodium perborate) as preservative. |
| Visine tears | 0.2% HPMC, 0.2% glycerin, 1% polyethylene glycol 400, |
| | benzalkonium chloride (0.01%) as preservative. Visine is also |
| | available as preservative-free single-use vials. |

Glycerin Artificial Tears & Others

| | |
|---|---|
| Advanced Eye Relief Dry Eye | 1% Glycerin, benzalkonium chloride (0.01%) as preservative. |
| Enironmental | |
| Computer Eye Drops | 1% Glycerin, benzalkonium chloride (0.01%) as preservative. |
| Moisture Eyes | 1% Propylene glycol, 0.3%, glycerin. Benzalkonium chloride |
| | (0.01%) as preservative. |
| Similasan Dry Eye Relief | Belladonna HPUS 6X alkaloids, Euphrasia, Mercurius |
| | sublimatus. Sodium silver chloride complex 0.001% as |
| | preservative. |
| Soothe Lubricating Eye Drops | Glycerin (0.6%)—lubricant; propylene glycol |
| | (0.6%)—lubricant, boric acid, hydroxyalkylphosphonate, |
| | purified water, sodium alginate, sodium borate. |

Polyvinyl Alcohol (PVA) Artifical Tears

| | |
|---|---|
| Murine Tears | 0.5% polyvinyl alcohol, 0.6% Povidone (has bicarbonate), |
| | benzalkonium chloride (0.01%) as preservative. Manufactured |
| | by Ross (Abbott Labs). |
| Hypotears | 1% polyvinyl alcohol, 1% polyethylene glycol 400. Hypotonic |
| | Benzalkonium chloride (0.01%) as preservative. Hypotears is |
| | also available as preservative-free single-use vials. |
| Akwa Tears | 1.4% polyvinyl alcohol. Hypotonic. Benzalkonium chloride |
| | (0.005%) as preservative. |
| Tears Again | 1.4% polyvinyl alcohol. |

Oil Containing Eye Drops-Prevent Evaporation of Tears

| | |
|---|---|
| Refresh Endura | Castor oil, polysorbate-80, carbomer 1342 & glycerin. |
| | Preservative-free. |
| SootheXP Emollient Eye Drops | Light mineral oil 1.0%, mineral oil 4.5% (Restoryl), |
| | Polysorbate-80, polyhexamethylene biguanide as preservative. |

Contact Lens Rewetting Drops (can be used while wearing soft or hard/gas permeable contact lens)

| | |
|---|---|
| Clerz Plus | Tetronic 1304 helps lens retain moisture & shields from protein |
| | build up. RLM-100 removes lens deposits. Polyquad is the |
| | preservative. |
| Aquify Comfort Drops | Sodium hyaluronate 0.1%, sodium perborate is the preservative. |
| Complete Blink-N-Clean | Hydroxypropyl methylcellulose is the lubricant, Tyloxapol is |
| | the surfactant. Polyhexamethylene biguanide is the |
| | preservative. |
| Opti-Free Express Rewetting | Sterile, buffered, isotonic, aqueous solution. No hydrogel |
| drops | lubricants. Polyquad is the preservative. |
| Complete Lubricating and | Sterile, isotonic, buffered, preserved solution. Hydroxypropyl |
| Rewetting Drops | methylcellulose is the lubricant, Tyloxapol is the surfactant. |
| Blink Contacts | Hyaluronate 0.15%, peroxide-free, non-sensitizing Ocupure ™ |
| | preservative. |
| ReNu Rewetting drops | Poloxamine removes dirt & protein deposits. Sorbic acid is the |
| | preservative. |

Eye Ointments & Gels

| | |
|---|---|
| Refreh PM ointment | 57.3% white petrolatum, 42.5% mineral oil, preservative-free. |
| Tears Naturale PM ointment | 56.8% white petrolatum, 42.5% mineral oil, preservative-free. |
| Refresh Lacri-lube ointment | White petrolatum, mineral oil, chlorobutanol (0.5%) as |
| | preservative. Manufactured by Allergan. |
| Genteal Gel | 0.3% hydroxypropyl methylcellulose, Carbopol 980, GenAqua |
| | (sodium perborate) as preservative. |
| Tears Again NIGHT & DAY Gel | 1.5% carboxymethylcellulose. Dissipate (stabilized oxyborate |
| | complex) as a preservative. |

### Example 3

### Mouse Dry Eye Model

This study validates a mouse model of dry eye described in IOVS 2006 47:133. Rimabotulinum toxin B (BTX-B) (Elan Pharmaceuticals) is injected into a mouse lacrimal gland, which attenuates tear secretion for up to a month and creates a clinical picture of dry eye that mimics that in humans. Specifically, on day 0, female CBA/J mice receive a 50 μl injection of saline containing 20 mU of BTX-B directly into the lacrimal gland of one eye. Control mice receive an injection of physiologic saline into the lacrimal gland of one eye. The eyes are evaluated for tear formation and corneal fluorescein staining (a measure of corneal ulcerations) on days 14 and 28. Relative to the saline injected mice, the BTX-B injected mice display a statistically significant, time dependent decrease in tear production and a statistically significant time dependent increase in fluorescein staining of the cornea. The data indicate that this model may be used to evaluate therapies for dry eye including, for example, P2X7 receptor antagonists and enzymes that degrade ATP.

### Example 4

### Evaluation of Eye Drops Containing a P2X7 Receptor Antagonist in the Mouse Dry Eye Model

This first study evaluates the efficacy of the potent and selective P2X7 receptor antagonist, A-740003, in the mouse BTX-B model of dry eye. Follow-up studies, below, evaluate additional P2X7 receptor inhibitors and apyrase in the same model.

A-740003 is purchased from Tocris Bioscience, Ellisville, Mo. The molecular weight is 475 daltons, and the IC₅₀ for the mouse P2X7 receptor is approximately 1 μM (British Journal of Pharmacology 2009 157:1203).

A 100 mM stock solution of A-740003 is prepared in DMSO. The stock solution is diluted into Allergan Refresh Celluvisc Eye Drops (see table above) to concentrations ranging from 10 nM to 100 μM (British Journal of Pharmacology 2009 157:1203).

Female CBA mice are injected on day 0 in one lacrimal gland with BTX-B. The mice are followed for 28 days. Tear production and corneal staining are evaluated on days 14 and 28. Starting on day 0, the mice receive eye drop treatments with 50 μl per eye three times per day. The eye drops are administered at doses of 0 (Refresh Celluvisc vehicle only), 10 nM, 100 nM, 1 μM, 10 μM, and 100 μM.

The results demonstrate that this experimental strategy can be used to identify a dose(s) at which the A-730003-treated mice exhibit significantly more tear production and significantly less corneal staining than the cohort that receives vehicle only.

### Example 5

### Evaluation of Eye Drops Containing a P2X7 Receptor Antagonist or Apyrase in the Mouse Dry Eye Model

The study above is repeated with other P2X7 receptor antagonists, A-438079 (Tocris Bioscience, Ellisville, Mo.), BBG, and oxATP (Sigma Aldrich, St. Louis, Mo.), and with apyrase (Sigma Aldrich, St. Louis, Mo.).

Stock solutions of each are prepared in water at the following concentrations for dilution into Allergan Refresh Celluvisc: A-438079, 5 mM; BBG, 10 mM; oxATP, 10 mM; and apyrase, 200 U/ml.

The doses tested for A-438079 are the same as those for A-730003, above. The doses tested for BBG are 100 nM, 1 μM, 10 μM, and 100 μM. The doses tested for oxATP are 100 nM, 1 μM, 10 μM, 100 μM, and 1 mM. Apyrase is tested in doses that range from 0.01 U/ml to 100 U/ml.

The data indicate that this experimental strategy can be used to identify doses at which the P2X7 receptor antagonists and apyrase are efficacious in attenuating pathology in this model.

### Example 6

### Evaluation of Eye Drops Containing a P2X7 Receptor Antagonist or Apyrase in a Mouse Model of Endotoxin-Mediated Keratoconjunctivitis and Uveitis

This study uses a mouse model of endotoxin induced keratoconjunctivitis and uveitis. C57BL/6 and C3H/HeN mice each receive an injection of 300 μg of Salmonella typhimurium endotoxin in a hind paw. Eighteen hours later, the animals exhibit keratoconjunctivitis with sloughing of the ocular surface epithelial cells and a thick discharge. The animals also develop uveitis in both the anterior and posterior segments of each eye. Uveitis is quantified by microscopic examination of H&E stained histopathology slides, and the numbers of inflammatory cells in the anterior and posterior segments of the eye are determined.

Eye drops containing the P2X7 receptor antagonists and apyrase are prepared as above. Vehicle alone eye drops serve as the control. Starting at the time of endotoxin injection, and for the ensuing 12 hours, the mice receive eye drops every three hours. The animals are evaluated 18 hours after injection for the quantity and cellularity of ocular discharge as well as for the numbers of inflammatory cells in the anterior and posterior segments of each eye.

The data indicate that this experimental strategy can be used to identify doses at which the P2X7 receptor antagonists and apyrase are efficacious in diminishing the amount and cellularity of the discharge and the numbers of inflammatory cells within each eye.

### Example 7

### Evaluation of Eye Drops Containing a P2X7 Receptor Antagonist or Apyrase in a Mouse Model of Corneal Neovascularization

This study uses an injury model of corneal neovascularization in BALB/c mice. Two silk sutures are placed near the center of each cornea. During the ensuing week, new blood vessels sprout from the limbic vessels, which are located around the periphery of the cornea, and grow up over the cornea towards the sutures. The blood vessels can be readily visualized against the albino background, and can be photographed and measured.

Eye drops containing the P2X7 receptor antagonists and apyrase are prepared as above. Vehicle alone eye drops serve as the control. Starting at the time of suture placement, the mice receive eye drops three times per day. After one week, the corneas are photographed and the number and length of new vessels is determined.

The data indicate that this experimental strategy can be used to identify doses at which the P2X7 receptor antagonists and apyrase are efficacious in diminishing the extent of new blood vessel growth.

### Example 8

### Evaluation of Eye Drops Containing a P2X7 Receptor Antagonist or Apyrase in a Mouse Model of Allergic Conjunctivitis

This study utilizes a model in which mice are first systemically immunized with ovalbumin and then challenged with ocular surface application of ovalbumin. The model is summarized in Allergy 2003 58:1101. C57BL/6 mice are immunized on day 0 with 100 μg ovalbumin/1 mg Alum/300 μg pertussis toxin via intraperitoneal injection. The mice are boosted on day 4 with 50 μg ovalbumin via subcutaneous injection and then challenged on day 17 with 750 μg ovalbumin via conjunctival administration. The mice are examined 24 hours later for clinical signs of conjunctivitis including conjunctival and lid erythema and edema. The conjunctiva is examined histologically for mast cell and eosinophil infiltration.

To assess the potential efficacy of P2X7 receptor antagonism and ATP degradation, the eye drop compositions described above are administered four times per day throughout the study starting on day 0. Additional cohorts receive eye drop treatments starting at the time of the ovalbumin challenge on day 17.

The data indicate that this experimental strategy can be used to identify doses at which the P2X7 receptor antagonists and apyrase are efficacious in diminishing the clinical signs of conjunctivitis as well as the cellular infiltrates.

### Example 9

### An Ophthalmically Acceptable Formulation Containing 1 μM Brilliant Blue G (BBG)

BSS Sterile Irrigating Solution is obtained from Alcon Laboratories, Ft. Worth, Tex. BBG is obtained from Sigma Aldrich, St. Louis, Mo. With moderate stirring in a homomixer, 856 mg of BBG is added to 1 liter of BSS at room temperature. The mixture is then stirred until the BBG is completely in solution. This solution is a 1 mM stock solution. One ml of the stock solution is then added to one liter of BSS to yield the ophthalmic composition, which is 1 μM BBG in BSS. The ophthalmic composition is filtered through a membrane filter (0.45 μm pore diameter, Millipore), filled in a 15 mL polyethylene terephthalate eye drop container, a polypropylene container, a polyethylene container, and a glass container and preserved at 60° C. for 2 days. The amount of BBG from each container is measured before and after preservation. The active ingredient shows little degradation.

### Example 10

### Artificial Lacrimal Fluid Containing 1 μM BBG

An artificial lacrimal fluid of the following formulation is prepared by a conventional method comprising potassium L-aspartate, 5 g; aminoethylsulfonic acid, 5 g; sodium chloride, 5 g; potassium chloride, 15 g; boric acid, 3 g; borax q.s. (pH); benzalkonium chloride, 0.05 g; 1 ml of a 1 mM stock solution of BBG in purified water, and purified water q.s. for a total amount of 1,000 ml (pH 7.2).

### Example 11

### Contact Lens Ophthalmic Solution Containing 1 μM BBG

A contact lens ophthalmic solution of the following formulation is prepared according to a conventional method and contains sodium chloride, 5.5 g; potassium chloride, 1.5 g; glucose, 0.05 g; boric acid, 6 g; borax q.s. (pH); potassium sorbate, 1 g; Macrogol 4000, 3 g; sodium edentate, 1 g; 1 ml of a 1 mM stock solution of BBG in purified water; and purified water q.s. for a total amount of 1,000 ml (pH 6.5).

### Example 12

### Ophthalmic Composition Combining Antihistamine with 1 μM BBG

An ophthalmic solution of the following formulation is prepared according to a conventional method and contains panthenol, 1 g; potassium L-aspartate, 10 g; allantoin, 1 g; chlorpheniramine maleate, 0.3 g; sodium chloride, 4.5 g; sodium L-glutamate, 2 g; polyoxyethylene hydrogenated castor oil 60, 3 g; hydrochloric acid, q.s. (pH); benzalkonium chloride, 0.05 g; chlorobutanol, 2 g; 1 ml of a 1 mM stock solution of BBG in purified water; and purified water q.s. for a total amount of 1,000 ml (pH 5.5).

### Example 13

### Lubricating Eye Drops Containing 1 μM BBG

A 0.8% sodium polyacrylate solution is prepared by adding 0.8 g of a straight chain-type sodium polyacrylate [trade name: Aronbis MS manufactured by Nihon Junyaku Co., Ltd., viscosity-average molecular weight: 2,000,000 to 3,000,000] to 99.2 g of an aqueous sodium chloride solution, which is obtained by dissolving 0.4 g of sodium chloride in 98.8 g of sterile distilled water, and dissolving the straight chain-type sodium polyacrylate using a stirrer.

Two (2) g of D-mannitol are added to 40 g of a 2.5 μM aqueous solution of BBG and dissolved thoroughly using a stirrer, and then 50 g of the 0.8% sodium polyacrylate solution and 8 g of sterile distilled water are added. The mixture is mixed by sufficient stirring to obtain a water-soluble eye drop. The pH is 8.0 and the viscosity at 25° C. is 20 cP.

### Example 14

### Oxidized ATP Completely Prevented Corneal Ulcerations in the Mouse Dry Eye Model

This study utilized the mouse dry eye model of Example 3 to evaluate one concentration of each of three P2X7 inhibitors administered as eye drops twice daily. The three P2X7 inhibitors were: 1) A-438079, a reversible, competitive inhibitor used at a final concentration of 20 μM; 2) BBG, a reversible, non-competitive inhibitor used at a final concentration of 20 μM; and 3) oxATP, an irreversible inhibitor used at a final concentration of 300 μM.

The formulation in which each inhibitor was dissolved was Allergan Refresh Celluvisc, a non-prescription dry eye product obtained from a pharmacy. The formulation contained NaCl, KCl, CaCl₂, Na Lactate, and 1% carboxymethyl cellulose, pH 6.

50× stock solutions of each inhibitor were prepared in physiologic saline:

for 1 mM A-438079, 3.4 mg were dissolved in 10 ml saline;

for 1 mM BBG, 9.5 mg were dissolved in 10 ml saline; and

for 15 mM oxATP, 18 mg were dissolved in 2 ml saline.

Each stock solution was filter sterilized. Then 0.1 ml of each stock was thoroughly mixed with 4.9 ml of Refresh Celluvisc. The formulation only control was prepared by mixing 0.1 ml saline with 4.9 ml Refresh Celluvisc. The test articles were aliquoted in sterile polypropylene tubes and stored frozen.

The study used adult, 8-10 week old male CBA/J mice. The mice were maintained in a 12 hour light/dark cycle. Prior to the study, both eyes of each mouse were examined to ensure no gross abnormalities. Each mouse was anesthetized with ketamine and xylazine, and the lacrimal glands of both eyes were injected with 50 μl of saline containing 20 mU of BTX-B.

The mice were separated into five cohorts:

1. no eye drop treatment;

2. formulation only eye drops twice daily;

3. A-438079 eye drops twice daily;

4. BBG eye drops twice daily; and

5. oxATP eye drops twice daily.

Fluorescein staining was used to quantify corneal ulceration, a measure of dry eye pathology. Each mouse was assessed by fluorescein staining prior to BTX-B injection (day 0) and 7 and 14 days after BTX-B injection. The staining was graded on the 0 to 4 scale shown in Table 2.

TABLE 2
Grading Scale for Fluorescein Staining

| | |
|---|---|
| Absence of fluorescein staining. | 0 |
| Slight fluorescent staining confined to a small focus. With diffuse | 1 |
| illumination, the underlying structures are easily visible. The outline of | |
| the papillary margin is as if there were no fluorescein staining. | |
| Moderate fluorescein staining confined to a small focus. With diffuse | 2 |
| illumination, the underlying structures are clearly visible, although there | |
| is some loss of detail. | |
| Marked fluorescein staining. Staining may involve a larger portion of the | 3 |
| cornea. With diffuse illumination, the underlying structures are barely | |
| visible but are not completely obliterated. | |
| Extreme fluorescein staining. With diffuse illumination, the underlying | 4 |
| structures cannot be observed. | |

Each eye was examined daily and abnormalities, if any, of the conjunctiva, cornea, iris, and lens were graded via pre-established scales. General clinical observations were performed daily, and the animals were weighed daily.

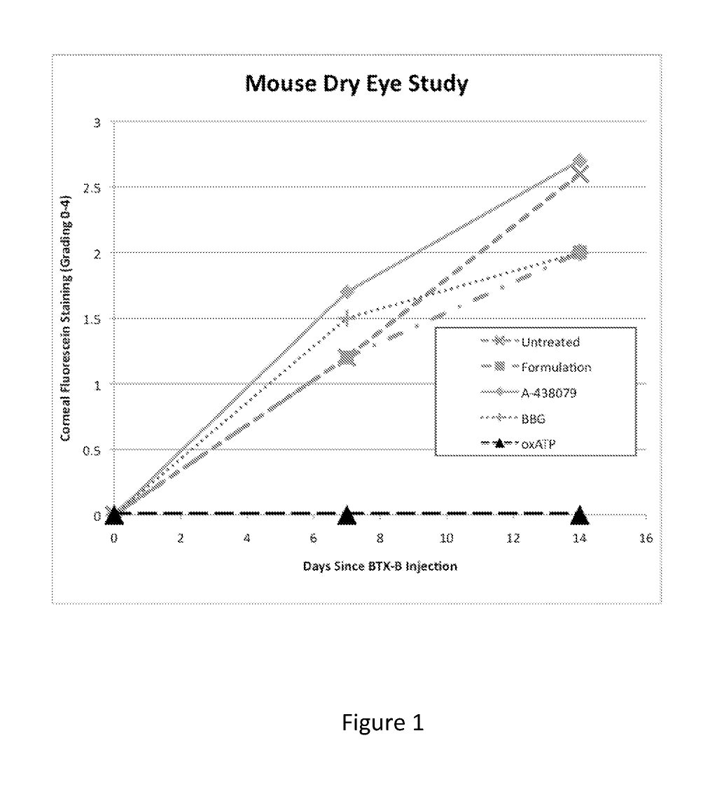

The results of the corneal fluorescein staining are shown in Table 3 and are depicted graphically in FIG. 1.

TABLE 3
Corneal Fluorescein Staining Results

| | DAY 0 | | | DAY 7 | | | DAY 14 | | |
|---|---|---|---|---|---|---|---|---|---|
| COHORT | MEAN | STDev | n | MEAN | STDev | n | MEAN | STDev | n |
| Untreated | 0.00 | 0.00 | 6 | 1.20 | 0.45 | 5 | 2.60 | 0.55 | 5 |
| Formulation | 0.00 | 0.00 | 6 | 1.20 | 0.45 | 5 | 2.00 | 0.00 | 5 |
| A-438079 | 0.00 | 0.00 | 4 | 1.70 | 0.58 | 3 | 2.70 | 0.58 | 3 |
| BBG | 0.00 | 0.00 | 6 | 1.50 | 0.58 | 4 | 2.00 | 0.00 | 4 |
| oxATP | 0.00 | 0.00 | 8 | 0.00 | 0.00 | 7 | 0.00 | 0.00 | 7 |

The ocular examinations, clinical observations, and animal weights were all normal, without any signs of toxicity.

No fluorescein staining was observed in any animal prior to the BTX-B mediated induction of dry eye. The staining pattern in the untreated cohort verified that the model behaved as expected, with increasing staining on days 7 and 14. At the chosen dose and b.i.d. dosing schedule, A-438079 and BBG did not attenuate fluorescein staining. In marked contrast, the oxATP eye drops completely prevented the fluorescein staining. Specifically, no fluorescein staining was seen at either the 7 or 14 day time point in any eye treated with oxATP. Thus, the oxATP eye drops completely prevented corneal ulceration and were highly effective in treating dry eye.

While some of the embodiments of the present invention have been described in detail in the above, those of ordinary skill in the art can enter various modifications and changes to the particular embodiments shown without substantially departing from the novel teaching and advantages of the present invention. Such modifications and changes are encompassed in the spirit and scope of the present invention as set forth in the appended claims.

All references cited herein are herein incorporated by reference in entirety.

1. A composition for application to an eye comprising an irreversible P2X7 inhibitor, oxATP, or a chemical derivative of oxATP that is an irreversible P2X7 inhibitor, and an ophthalmically acceptable formulation.

2. The composition of claim 1, comprising an aqueous solution, cream, or ointment.

3. The composition of claim 2, further comprising a container comprising a nozzle or eye dropper.

4. The composition of claim 1, further comprising a drug delivery depot.

5. The composition of claim 4, wherein said drug delivery depot comprises an in situ-formed gel, a collagen corneal shield, a drug delivery device, or a contact lens.

6. The composition of claim 1, wherein said irreversible P2X7 inhibitor is also an inhibitor of ecto-ATPase.

7. A method comprising topically administering to a patient with an eye disease associated with aberrant ATP metabolism, aberrant purinergic signaling or both, the composition of claim 1.

8. The method of claim 7, wherein said eye disease is a disorder of the cornea and conjunctiva (DOCC).

9. The method of claim 8, wherein said DOCC is dry eye or uveitis.

10. A medicament to treat an eye disease comprising the composition of claim 1 and a pharmaceutically acceptable carrier, diluent or excipient.

11. The medicament of claim 10, wherein said eye disease is a DOCC.

12. The medicament of claim 11. wherein said DOCC is dry eye or uveitis.

13. The medicament of claim 10, further comprising a container with a nozzle or eye dropper.

14. The medicament of claim 10, further comprising a drug delivery depot.

15. The medicament of claim 14, wherein said drug delivery depot comprises an in situ-formed gel, a collagen corneal shield, a drug delivery device, or a contact lens.

16.-18. (canceled)

19. A composition for application to an eye comprising a reversible P2X7 inhibitor, and an ophthalmically acceptable formulation packaged in a container comprising a nozzle or eye dropper.

20. (canceled)
